SEQUENCE LISTING (22 sequences)

SEQ ID NO: 1 - DNA, 3350 bp, Murinae gen. sp.
ccatcctggtctatagagagagttccagaacagccagggctacagataaacccatctggaaaaacaaagttgaatgacccaagaggggttctcagagggtggcgtgtgctccctggcaagcctatgacatggccggggcctgcctctctctgcctctgaccctcagtggctcccatgaactccttgcccaatggcatctttttcctgcgctccttgggttattccagtctcccctcagcattccttcctcagggcctcgctcttctctctgctccctccttgcacagctggctctgtccacctcagatgtcacagtgctctctcagaggaggaaggcaccatgtaccctctgtttcccaggtaagggttcaatttttaaaaatggttttttgtttgtttgtttgtttgtttgtttgtttgtttttcaagacagggctcctctgtgtagtcctaactgtcttgaaactccctctgtagaccaggtcgacctcgaactcttgaaacctgccacggaccacccagtcaggtatggaggtccctggaatgagcgtcctcgaagctaggtgggtaagggttcggcggtgacaaacagaaacaaacacagaggcagtttgaatctgagtgtattttgcagctctcaagcaggggattttatacataaaaaaaaaaaaaaaaaaaaaaccaaacattacatctcttagaaactatatccaatgaaacaatcacagataccaaccaaaaccattgggcagagtaaagcacaaaaatcatccaagcattacaactctgaaaccatgtattcagtgaatcacaaacagaacaggtaacatcattattaatataaatcaccaaaatataacaattctaaaaggatgtatccagtgggggctgtcgtccaaggctagtggcagatttccaggagcaggttagtaaatcttaaccactgaactaactctccagccccatggtcaattattatttagcatctagtgcctaatttttttttataaatcttcactatgtaatttaaaactattttaattcttcctaattaaggctttctttaccatataccaaaattcacctccaatgacacacgcgtagccatatgaaattttattgttgggaaaatttgtacctatcataatagttttgtaaatgatttaaaaagcaaagtgttagccgggcgtggtggcacacgcctttaatccctgcactcgggaggcaggggcaggaggatttctgagtttgaggccagcctggtctacagagtgagttccaggacagccagggctacacagagaaaccctgtctcgaaccccccaccccccaaaaaaagcaaagtgttggtttccttggggataaagtcatgttagtggcccatctctaggcccatctcacccattattctcgcttaagatcttggcctaggctaccaggaacatgtaaataagaaaaggaataagagaaaacaaaacagagagattgccatgagaactacggctcaatattttttctctccggcgaagagttccacaaccatctccaggaggcctccacgttttgaggtcaatggcctcagtctgtggaacttgtcacacagatcttactggaggtggtgtggcagaaacccattccttttagtgtcttgggctaaaagtaaaaggcccagaggaggcctttgctcatctgaccatgctgacaaggaacacgggtgccaggacagaggctggaccccaggaacaccttaaacacttcttcccttctccgccccctagagcaggctcccctcaccagcctgggcagaaatgggggaagatggagtgaagccatactggctactccagaatcaacagagggagccgggggcaatactggagaagctggtctccccccaggggcaatcctggcacctcccaggcagaagaggaaacttccacagtgcatctcacttccatgaatcccctcctcggactctgaggtccttggtcacagctgaggtgcaaaaggctcctgtcatattgtgtcctgctctggtctgccttccacagcttgggggccacctagcccacctctccctagggatgagagcagccactacgggtctaggctgcccatgtaaggaggcaaggcctggggacacccgagatgcctggttataattaacccagacatgtggctgcccccccccccccaacacctgctgcctgagcctcacccccaccccggtgcctgggtcttaggctctgtacaccatggaggagaagctcgctctaaaaataaccctgtccctggtggatccagggtgaggggcaggctgagggcggccacttccctcagccgcaggtttgttttcccaagaatggtttttctgcttctgtagcttttcctgtcaattctgccatggtggagcagcctgcactgggcttctgggagaaaccaaaccgggttctaacctttcagctacagttattgcctttcctgtagatgggcgactacagccccacccccacccccgtctcctgtatccttcctgggcctggggatcctaggctttcactggaaatttccccccaggtgctgtaggctagagtcacggctcccaagaacagtgcttgcctggcatgcatggttctgaacctccaactgcaaaaaatgacacataccttgacccttggaaggctgaggcagggggattgccatgagtgcaaagccagactgggtggcatagttagaccctgtctcaaaaaaccaaaaacaattaaataactaaagtcaggcaagtaatcctactcgggagactgaggcagagggattgttacatgtctgaggccagcctggactacatagggtttcaggctagccctgtctacagagtaaggccctatttcaaaaacacaaacaaaatggttctcccagctgctaatgctcaccaggcaatgaagcctggtgagcattagcaatgaaggcaatgaaggagggtgctggctacaatcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccccc

SEQ ID NO: 2 - DNA, 53 bp, Murinae gen. sp.
gggtcaccaccacctccacagcacagacagacactcaggagccagccagccag

SEQ ID NO: 3 - DNA, 2972 bp, Murinae gen. sp.
gtagggactgagagaaatcactggggtgggagtggggcgtgggagtccaagggtctgctcacccagtcatgttatggttgtggattttgcagcacaagttgtggggacaaatgtctgggacacctaggtctcaatagccaccaagtgtcccctccttgcaaggcagggtgggctggaacttagtttagcagagttaatggcccacacaaagacagttgtctcagtgacacctgtcagtggccctttaactttgtaaccatgtggacctgtgttgcagctctgtgaccttgtgtctcactgtcctggtctgtctctatgtctctctgtctctctgtctctatctctctctttctgtctctctctctccctctctctttcgagatgggtcaggggggggtggtgttctctgcatagccctggctgtcctggaactcactctgtagaccagcctggcctcgaactcagaaatccacctgcctcccaagtgctgggattaaaggcgtgtgccaccaccgcccggcgggtctttcttgtgtgagacttgggggctctcactcttacaggcccctggctttcctttgagtccttctgtctggctgtctctgggatcttgaaggcaggaaggactacatgactcagtttacctggagatcttagagaatctgtgatgagtttggggattccgaagctttctgcttctgcgtcttgcctcggtgtcctgtctcctggggtgcccctgagggagggggtagcagaggatacagaaccttctgaagggagagatctgggctgggagcccggggtgtccttgaggcccagagcctggctgtgtgtcctcctggccaccccagcccacctgtcccaatgctgacttagtgcaaggcgagccagcaaggagggaggacaggtggcagtggggggtgaggagcatctaaaaatagccacaaagtagcagcttcaagggctttgggtctctgtctgccccacactcttctctcagcttggtccaccttccctctcaccttcctctgaggcccccttccagccccgatggaggcctgatgtcccccatggtcagtgcttcagggatctagtcaataaaattaataatgaaaaacaacagtaataaaatacacgtgacgtgactggggcagcttagggcttagttcaaatcccagtgttcacaccctttaaaagacaagacaaaacaaaacagctggctgtgggggagaacatcagaatccccctggggaggtggggacaggggatctgtggggctccatggccagccagcctagctccaggcctgcgagagaccctacctcaagataaaaataaaataaaataaaataaatatataaaataacaatcttgcagcacctgaggtcaccactggaatgtgcacacctgtgcacatacatgagcctgcactacaaacaaaaatattaacagtaactgttagaatcccagctgcaacttcatgccaggtgccaggtccatgctcatcagtcagggactggaactcagagatctcctgggaaagcttcagtctcacagattcaaaagccagagagatctagtcacagcctggggcccagagcagtgacttaggagagccgtgccttttaaagtggaccttgtagacagccagaggtggagggactgggagaagtggctgaagcctccagactcattcccacgcccacatctggactaatttggatcagaatctcaggggagcccttatggcttttctcaggtgtgcacatataatctttaccagggtcctcacacagagcctgtcagattggttttcaatttctgtgacaaacaccatgaccaagacaacctagaaaagagaaagcattaatttggggctcagggttctggagcggcagggaggtgggcatggtgctggagcagaggctggaagctcacatctttatcaacaaccagaggcagtgagagccacttgggaatggggtggcttttcggaaatctcaaagcccacaagcaatggcacacctcctccaacaaggccacacctccgaatccttcccaaacagttccaccgactggggaccaaacattcaaatatgtgagtctgaggctcttctcattcaaatcaccacagacccaagaacaatcgaataaaatatttgtgttatgtgccaggcactggccgaggcgcttttcttgtcttttaatccctcccaagaggtcagcgatgccacagtctccatgttacagatgagtgaacaggaaagtcaaacaggctcctcagagtcacgcggctgcttgtaagttgcaaagccgaaattcgaacccagaccatctgatccagatcctttgctgcttttattcatctttttattttattttattttattttaattcctggtggcagggtttctgtagcccaggctacccttgaattcactgcaatcctcctgcctcagtttcagagtgttggaattacaagcatggaccatcatgcccagttcctttgggttgagatagagacctgtgtaggagcccagactcgggctggtctccagctctctacgtagatgaagatgaccttgaactgctgggatttcaggcatgagcagccacacccagatttgctgagcgccaaactgttacccagggtcctaagcttgctgggcaagcactctgccagcagaaccccagccccagatcctgtatttttgtagttgtttttgtttatgtgactgtccttttctggctttagacaaaaggttttgccctccttttccagctagagagactgagtccccagcaggatcacataggcaggatgtggccacatcaggcaacttgggctcctgatgtttccttgcaaggctgaggttcacagggggagaacccccctttttcaagcccacggtccgacggactgcaagcccccagcaactgagttcttaagtctgaaccctttcttctcacag

SEQ ID NO: 4 - DNA, 16 bp, Murinae gen. sp.
ggtcccaaaggccgcc

SEQ ID NO: 5 - DNA, 6375 bp, Murinae gen. sp.
ccatcctggtctatagagagagttccagaacagccagggctacagataaacccatctggaaaaacaaagttgaatgacccaagaggggttctcagagggtggcgtgtgctccctggcaagcctatgacatggccggggcctgcctctctctgcctctgaccctcagtggctcccatgaactccttgcccaatggcatctttttcctgcgctccttgggttattccagtctcccctcagcattccttcctcagggcctcgctcttctctctgctccctccttgcacagctggctctgtccacctcagatgtcacagtgctctctcagaggaggaaggcaccatgtaccctctgtttcccaggtaagggttcaatttttaaaaatggttttttgtttgtttgtttgtttgtttgtttgtttgtttttcaagacagggctcctctgtgtagtcctaactgtcttgaaactccctctgtagaccaggtcgacctcgaactcttgaaacctgccacggaccacccagtcaggtatggaggtccctggaatgagcgtcctcgaagctaggtgggtaagggttcggcggtgacaaacagaaacaaacacagaggcagtttgaatctgagtgtattttgcagctctcaagcaggggattttatacataaaaaaaaaaaaaaaaaaaaaaccaaacattacatctcttagaaactatatccaatgaaacaatcacagataccaaccaaaaccattgggcagagtaaagcacaaaaatcatccaagcattacaactctgaaaccatgtattcagtgaatcacaaacagaacaggtaacatcattattaatataaatcaccaaaatataacaattctaaaaggatgtatccagtgggggctgtcgtccaaggctagtggcagatttccaggagcaggttagtaaatcttaaccactgaactaactctccagccccatggtcaattattatttagcatctagtgcctaatttttttttataaatcttcactatgtaatttaaaactattttaattcttcctaattaaggctttctttaccatataccaaaattcacctccaatgacacacgcgtagccatatgaaattttattgttgggaaaatttgtacctatcataatagttttgtaaatgatttaaaaagcaaagtgttagccgggcgtggtggcacacgcctttaatccctgcactcgggaggcaggggcaggaggatttctgagtttgaggccagcctggtctacagagtgagttccaggacagccagggctacacagagaaaccctgtctcgaaccccccaccccccaaaaaaagcaaagtgttggtttccttggggataaagtcatgttagtggcccatctctaggcccatctcacccattattctcgcttaagatcttggcctaggctaccaggaacatgtaaataagaaaaggaataagagaaaacaaaacagagagattgccatgagaactacggctcaatattttttctctccggcgaagagttccacaaccatctccaggaggcctccacgttttgaggtcaatggcctcagtctgtggaacttgtcacacagatcttactggaggtggtgtggcagaaacccattccttttagtgtcttgggctaaaagtaaaaggcccagaggaggcctttgctcatctgaccatgctgacaaggaacacgggtgccaggacagaggctggaccccaggaacaccttaaacacttcttcccttctccgccccctagagcaggctcccctcaccagcctgggcagaaatgggggaagatggagtgaagccatactggctactccagaatcaacagagggagccgggggcaatactggagaagctggtctccccccaggggcaatcctggcacctcccaggcagaagaggaaacttccacagtgcatctcacttccatgaatcccctcctcggactctgaggtccttggtcacagctgaggtgcaaaaggctcctgtcatattgtgtcctgctctggtctgccttccacagcttgggggccacctagcccacctctccctagggatgagagcagccactacgggtctaggctgcccatgtaaggaggcaaggcctggggacacccgagatgcctggttataattaacccagacatgtggctgcccccccccccccaacacctgctgcctgagcctcacccccaccccggtgcctgggtcttaggctctgtacaccatggaggagaagctcgctctaaaaataaccctgtccctggtggatccagggtgaggggcaggctgagggcggccacttccctcagccgcaggtttgttttcccaagaatggtttttctgcttctgtagcttttcctgtcaattctgccatggtggagcagcctgcactgggcttctgggagaaaccaaaccgggttctaacctttcagctacagttattgcctttcctgtagatgggcgactacagccccacccccacccccgtctcctgtatccttcctgggcctggggatcctaggctttcactggaaatttccccccaggtgctgtaggctagagtcacggctcccaagaacagtgcttgcctggcatgcatggttctgaacctccaactgcaaaaaatgacacataccttgacccttggaaggctgaggcagggggattgccatgagtgcaaagccagactgggtggcatagttagaccctgtctcaaaaaaccaaaaacaattaaataactaaagtcaggcaagtaatcctactcgggagactgaggcagagggattgttacatgtctgaggccagcctggactacatagggtttcaggctagccctgtctacagagtaaggccctatttcaaaaacacaaacaaaatggttctcccagctgctaatgctcaccaggcaatgaagcctggtgagcattagcaatgaaggcaatgaaggagggtgctggctacaatcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcaccaccacctccacagcacagacagacactcaggagccagccagccaggtagggactgagagaaatcactggggtgggagtggggcgtgggagtccaagggtctgctcacccagtcatgttatggttgtggattttgcagcacaagttgtggggacaaatgtctgggacacctaggtctcaatagccaccaagtgtcccctccttgcaaggcagggtgggctggaacttagtttagcagagttaatggcccacacaaagacagttgtctcagtgacacctgtcagtggccctttaactttgtaaccatgtggacctgtgttgcagctctgtgaccttgtgtctcactgtcctggtctgtctctatgtctctctgtctctctgtctctatctctctctttctgtctctctctctccctctctctttcgagatgggtcaggggggggtggtgttctctgcatagccctggctgtcctggaactcactctgtagaccagcctggcctcgaactcagaaatccacctgcctcccaagtgctgggattaaaggcgtgtgccaccaccgcccggcgggtctttcttgtgtgagacttgggggctctcactcttacaggcccctggctttcctttgagtccttctgtctggctgtctctgggatcttgaaggcaggaaggactacatgactcagtttacctggagatcttagagaatctgtgatgagtttggggattccgaagctttctgcttctgcgtcttgcctcggtgtcctgtctcctggggtgcccctgagggagggggtagcagaggatacagaaccttctgaagggagagatctgggctgggagcccggggtgtccttgaggcccagagcctggctgtgtgtcctcctggccaccccagcccacctgtcccaatgctgacttagtgcaaggcgagccagcaaggagggaggacaggtggcagtggggggtgaggagcatctaaaaatagccacaaagtagcagcttcaagggctttgggtctctgtctgccccacactcttctctcagcttggtccaccttccctctcaccttcctctgaggcccccttccagccccgatggaggcctgatgtcccccatggtcagtgcttcagggatctagtcaataaaattaataatgaaaaacaacagtaataaaatacacgtgacgtgactggggcagcttagggcttagttcaaatcccagtgttcacaccctttaaaagacaagacaaaacaaaacagctggctgtgggggagaacatcagaatccccctggggaggtggggacaggggatctgtggggctccatggccagccagcctagctccaggcctgcgagagaccctacctcaagataaaaataaaataaaataaaataaatatataaaataacaatcttgcagcacctgaggtcaccactggaatgtgcacacctgtgcacatacatgagcctgcactacaaacaaaaatattaacagtaactgttagaatcccagctgcaacttcatgccaggtgccaggtccatgctcatcagtcagggactggaactcagagatctcctgggaaagcttcagtctcacagattcaaaagccagagagatctagtcacagcctggggcccagagcagtgacttaggagagccgtgccttttaaagtggaccttgtagacagccagaggtggagggactgggagaagtggctgaagcctccagactcattcccacgcccacatctggactaatttggatcagaatctcaggggagcccttatggcttttctcaggtgtgcacatataatctttaccagggtcctcacacagagcctgtcagattggttttcaatttctgtgacaaacaccatgaccaagacaacctagaaaagagaaagcattaatttggggctcagggttctggagcggcagggaggtgggcatggtgctggagcagaggctggaagctcacatctttatcaacaaccagaggcagtgagagccacttgggaatggggtggcttttcggaaatctcaaagcccacaagcaatggcacacctcctccaacaaggccacacctccgaatccttcccaaacagttccaccgactggggaccaaacattcaaatatgtgagtctgaggctcttctcattcaaatcaccacagacccaagaacaatcgaataaaatatttgtgttatgtgccaggcactggccgaggcgcttttcttgtcttttaatccctcccaagaggtcagcgatgccacagtctccatgttacagatgagtgaacaggaaagtcaaacaggctcctcagagtcacgcggctgcttgtaagttgcaaagccgaaattcgaacccagaccatctgatccagatcctttgctgcttttattcatctttttattttattttattttattttaattcctggtggcagggtttctgtagcccaggctacccttgaattcactgcaatcctcctgcctcagtttcagagtgttggaattacaagcatggaccatcatgcccagttcctttgggttgagatagagacctgtgtaggagcccagactcgggctggtctccagctctctacgtagatgaagatgaccttgaactgctgggatttcaggcatgagcagccacacccagatttgctgagcgccaaactgttacccagggtcctaagcttgctgggcaagcactctgccagcagaaccccagccccagatcctgtatttttgtagttgtttttgtttatgtgactgtccttttctggctttagacaaaaggttttgccctccttttccagctagagagactgagtccccagcaggatcacataggcaggatgtggccacatcaggcaacttgggctcctgatgtttccttgcaaggctgaggttcacagggggagaacccccctttttcaagcccacggtccgacggactgcaagcccccagcaactgagttcttaagtctgaaccctttcttctcacag

SEQ ID NO: 6 - DNA, 200 bp, Murinae gen. sp.
ctgagtccccagcaggatcacataggcaggatgtggccacatcaggcaacttgggctcctgatgtttccttgcaaggctgaggttcacagggggagaacccccctttttcaagcccacggtccgacggactgcaagcccccagcaactgagttcttaagtctgaaccctttcttctcacagggtcccaaaggccgccaat

SEQ ID NO: 7 - DNA, 186 bp, Homo sapiens
atgagtgccaggaagctgcgaaatctgtcttacaaaaaggtgattgtggaagagtctagaatcttcatttattgttcagcaggattacagaaaagctatcaagagtaaacatttaactgatacactcttattccttctttttaggctgtaaggaggcaaaagttgcttgaacagagcatccagtct

SEQ ID NO: 8 - DNA, 229 bp, Bos taurus
acacgtgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagcggccgc

SEQ ID NO: 9 - DNA, 13997 bp, Homo sapiens
ccatcctggtctatagagagagttccagaacagccagggctacagataaacccatctggaaaaacaaagttgaatgacccaagaggggttctcagagggtggcgtgtgctccctggcaagcctatgacatggccggggcctgcctctctctgcctctgaccctcagtggctcccatgaactccttgcccaatggcatctttttcctgcgctccttgggttattccagtctcccctcagcattccttcctcagggcctcgctcttctctctgctccctccttgcacagctggctctgtccacctcagatgtcacagtgctctctcagaggaggaaggcaccatgtaccctctgtttcccaggtaagggttcaatttttaaaaatggttttttgtttgtttgtttgtttgtttgtttgtttgtttttcaagacagggctcctctgtgtagtcctaactgtcttgaaactccctctgtagaccaggtcgacctcgaactcttgaaacctgccacggaccacccagtcaggtatggaggtccctggaatgagcgtcctcgaagctaggtgggtaagggttcggcggtgacaaacagaaacaaacacagaggcagtttgaatctgagtgtattttgcagctctcaagcaggggattttatacataaaaaaaaaaaaaaaaaaaaaaccaaacattacatctcttagaaactatatccaatgaaacaatcacagataccaaccaaaaccattgggcagagtaaagcacaaaaatcatccaagcattacaactctgaaaccatgtattcagtgaatcacaaacagaacaggtaacatcattattaatataaatcaccaaaatataacaattctaaaaggatgtatccagtgggggctgtcgtccaaggctagtggcagatttccaggagcaggttagtaaatcttaaccactgaactaactctccagccccatggtcaattattatttagcatctagtgcctaatttttttttataaatcttcactatgtaatttaaaactattttaattcttcctaattaaggctttctttaccatataccaaaattcacctccaatgacacacgcgtagccatatgaaattttattgttgggaaaatttgtacctatcataatagttttgtaaatgatttaaaaagcaaagtgttagccgggcgtggtggcacacgcctttaatccctgcactcgggaggcaggggcaggaggatttctgagtttgaggccagcctggtctacagagtgagttccaggacagccagggctacacagagaaaccctgtctcgaaccccccaccccccaaaaaaagcaaagtgttggtttccttggggataaagtcatgttagtggcccatctctaggcccatctcacccattattctcgcttaagatcttggcctaggctaccaggaacatgtaaataagaaaaggaataagagaaaacaaaacagagagattgccatgagaactacggctcaatattttttctctccggcgaagagttccacaaccatctccaggaggcctccacgttttgaggtcaatggcctcagtctgtggaacttgtcacacagatcttactggaggtggtgtggcagaaacccattccttttagtgtcttgggctaaaagtaaaaggcccagaggaggcctttgctcatctgaccatgctgacaaggaacacgggtgccaggacagaggctggaccccaggaacaccttaaacacttcttcccttctccgccccctagagcaggctcccctcaccagcctgggcagaaatgggggaagatggagtgaagccatactggctactccagaatcaacagagggagccgggggcaatactggagaagctggtctccccccaggggcaatcctggcacctcccaggcagaagaggaaacttccacagtgcatctcacttccatgaatcccctcctcggactctgaggtccttggtcacagctgaggtgcaaaaggctcctgtcatattgtgtcctgctctggtctgccttccacagcttgggggccacctagcccacctctccctagggatgagagcagccactacgggtctaggctgcccatgtaaggaggcaaggcctggggacacccgagatgcctggttataattaacccagacatgtggctgcccccccccccccaacacctgctgcctgagcctcacccccaccccggtgcctgggtcttaggctctgtacaccatggaggagaagctcgctctaaaaataaccctgtccctggtggatccagggtgaggggcaggctgagggcggccacttccctcagccgcaggtttgttttcccaagaatggtttttctgcttctgtagcttttcctgtcaattctgccatggtggagcagcctgcactgggcttctgggagaaaccaaaccgggttctaacctttcagctacagttattgcctttcctgtagatgggcgactacagccccacccccacccccgtctcctgtatccttcctgggcctggggatcctaggctttcactggaaatttccccccaggtgctgtaggctagagtcacggctcccaagaacagtgcttgcctggcatgcatggttctgaacctccaactgcaaaaaatgacacataccttgacccttggaaggctgaggcagggggattgccatgagtgcaaagccagactgggtggcatagttagaccctgtctcaaaaaaccaaaaacaattaaataactaaagtcaggcaagtaatcctactcgggagactgaggcagagggattgttacatgtctgaggccagcctggactacatagggtttcaggctagccctgtctacagagtaaggccctatttcaaaaacacaaacaaaatggttctcccagctgctaatgctcaccaggcaatgaagcctggtgagcattagcaatgaaggcaatgaaggagggtgctggctacaatcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcaccaccacctccacagcacagacagacactcaggagccagccagccaggtagggactgagagaaatcactggggtgggagtggggcgtgggagtccaagggtctgctcacccagtcatgttatggttgtggattttgcagcacaagttgtggggacaaatgtctgggacacctaggtctcaatagccaccaagtgtcccctccttgcaaggcagggtgggctggaacttagtttagcagagttaatggcccacacaaagacagttgtctcagtgacacctgtcagtggccctttaactttgtaaccatgtggacctgtgttgcagctctgtgaccttgtgtctcactgtcctggtctgtctctatgtctctctgtctctctgtctctatctctctctttctgtctctctctctccctctctctttcgagatgggtcaggggggggtggtgttctctgcatagccctggctgtcctggaactcactctgtagaccagcctggcctcgaactcagaaatccacctgcctcccaagtgctgggattaaaggcgtgtgccaccaccgcccggcgggtctttcttgtgtgagacttgggggctctcactcttacaggcccctggctttcctttgagtccttctgtctggctgtctctgggatcttgaaggcaggaaggactacatgactcagtttacctggagatcttagagaatctgtgatgagtttggggattccgaagctttctgcttctgcgtcttgcctcggtgtcctgtctcctggggtgcccctgagggagggggtagcagaggatacagaaccttctgaagggagagatctgggctgggagcccggggtgtccttgaggcccagagcctggctgtgtgtcctcctggccaccccagcccacctgtcccaatgctgacttagtgcaaggcgagccagcaaggagggaggacaggtggcagtggggggtgaggagcatctaaaaatagccacaaagtagcagcttcaagggctttgggtctctgtctgccccacactcttctctcagcttggtccaccttccctctcaccttcctctgaggcccccttccagccccgatggaggcctgatgtcccccatggtcagtgcttcagggatctagtcaataaaattaataatgaaaaacaacagtaataaaatacacgtgacgtgactggggcagcttagggcttagttcaaatcccagtgttcacaccctttaaaagacaagacaaaacaaaacagctggctgtgggggagaacatcagaatccccctggggaggtggggacaggggatctgtggggctccatggccagccagcctagctccaggcctgcgagagaccctacctcaagataaaaataaaataaaataaaataaatatataaaataacaatcttgcagcacctgaggtcaccactggaatgtgcacacctgtgcacatacatgagcctgcactacaaacaaaaatattaacagtaactgttagaatcccagctgcaacttcatgccaggtgccaggtccatgctcatcagtcagggactggaactcagagatctcctgggaaagcttcagtctcacagattcaaaagccagagagatctagtcacagcctggggcccagagcagtgacttaggagagccgtgccttttaaagtggaccttgtagacagccagaggtggagggactgggagaagtggctgaagcctccagactcattcccacgcccacatctggactaatttggatcagaatctcaggggagcccttatggcttttctcaggtgtgcacatataatctttaccagggtcctcacacagagcctgtcagattggttttcaatttctgtgacaaacaccatgaccaagacaacctagaaaagagaaagcattaatttggggctcagggttctggagcggcagggaggtgggcatggtgctggagcagaggctggaagctcacatctttatcaacaaccagaggcagtgagagccacttgggaatggggtggcttttcggaaatctcaaagcccacaagcaatggcacacctcctccaacaaggccacacctccgaatccttcccaaacagttccaccgactggggaccaaacattcaaatatgtgagtctgaggctcttctcattcaaatcaccacagacccaagaacaatcgaataaaatatttgtgttatgtgccaggcactggccgaggcgcttttcttgtcttttaatccctcccaagaggtcagcgatgccacagtctccatgttacagatgagtgaacaggaaagtcaaacaggctcctcagagtcacgcggctgcttgtaagttgcaaagccgaaattcgaacccagaccatctgatccagatcctttgctgcttttattcatctttttattttattttattttattttaattcctggtggcagggtttctgtagcccaggctacccttgaattcactgcaatcctcctgcctcagtttcagagtgttggaattacaagcatggaccatcatgcccagttcctttgggttgagatagagacctgtgtaggagcccagactcgggctggtctccagctctctacgtagatgaagatgaccttgaactgctgggatttcaggcatgagcagccacacccagatttgctgagcgccaaactgttacccagggtcctaagcttgctgggcaagcactctgccagcagaaccccagccccagatcctgtatttttgtagttgtttttgtttatgtgactgtccttttctggctttagacaaaaggttttgccctccttttccagctagagagactgagtccccagcaggatcacataggcaggatgtggccacatcaggcaacttgggctcctgatgtttccttgcaaggctgaggttcacagggggagaacccccctttttcaagcccacggtccgacggactgcaagcccccagcaactgagttcttaagtctgaaccctttcttctcacagggtcccaaaggccgccaatatgagtgccaggaagctgcgaaatctgtcttacaaaaaggtgattgtggaagagtctagaatcttcatttattgttcagcaggattacagaaaagctatcaagagtaaacatttaactgatacactcttattccttctttttaggctgtaaggaggcaaaagttgcttgaacagagcatccagtctgcgcaggagactgaaaaatccttacacttaatccaggagtccctcacattcattgacaagcagttggcagcttatattgcagacaaggtggacgcagctcaaatgcctcaggaagcccagaaaatccaatctgatttgacaagtcatgagatcagtttagaagaaatgaagaaacataatcaggggaaggaggctgcccaaagagtcctgtctcagattgatgttgcacagaaaaaattacaagatgtctccatgaagtttcgattattccagaaaccagccaattttgagcagcgtctacaagaaagtaagatgattttagatgaagtgaagatgcacttgcctgcattggaaacaaagagtgtggaacaggaagtagtacagtcacagctaaatcattgtgtgaacttgtataaaagtctgagtgaagtgaagtctgaagtggaaatggtgataaagactggacgtcagattgtacagaaaaagcagacggaaaatcccaaagaacttgatgaaagagtaacagctttgaaattgcattataatgagctgggagcaaaggtaacagaaagaaagcaacagttggagaaatgcttgaaattgtcccgtaagatgcgaaaggaaatgaatgtcttgacagaatggctggcagctacagatatggaattgacaaagagatcagcagttgaaggaatgcctagtaatttggattctgaagttgcctggggaaaggctactcaaaaagagattgagaaacagaaggtgcacctgaagagtatcacagaggtaggagaggccttgaaaacagttttgggcaagaaggagacgttggtggaagataaactcagtcttctgaatagtaactggatagctgtcacctcccgagcagaagagtggttaaatcttttgttggaataccagaaacacatggaaacttttgaccagaatgtggaccacatcacaaagtggatcattcaggctgacacacttttggatgaatcagagaaaaagaaaccccagcaaaaagaagacgtgcttaagcgtttaaaggcagaactgaatgacatacgcccaaaggtggactctacacgtgaccaagcagcaaacttgatggcaaaccgcggtgaccactgcaggaaattagtagagccccaaatctcagagctcaaccatcgatttgcagccatttcacacagaattaagactggaaaggcctccattcctttgaaggaattggagcagtttaactcagatatacaaaaattgcttgaaccactggaggctgaaattcagcagggggtgaatctgaaagaggaagacttcaataaagatatgaatgaagacaatgagggtactgtaaaagaattgttgcaaagaggagacaacttacaacaaagaatcacagatgagagaaagagagaggaaataaagataaaacagcagctgttacagacaaaacataatgctctcaaggatttgaggtctcaaagaagaaaaaaggctctagaaatttctcatcagtggtatcagtacaagaggcaggctgatgatctcctgaaatgcttggatgacattgaaaaaaaattagccagcctacctgagcccagagatgaaaggaaaataaaggaaattgatcgggaattgcagaagaagaaagaggagctgaatgcagtgcgtaggcaagctgagggcttgtctgaggatggggccgcaatggcagtggagccaactcagatccagctcagcaagcgctggcgggaaattgagagcaaatttgctcagtttcgaagactcaactttgcacaaattcacactgtccgtgaagaaacgatgatggtgatgactgaagacatgcctttggaaatttcttatgtgccttctacttatttgactgaaatcactcatgtctcacaagccctattagaagtggaacaacttctcaatgctcctgacctctgtgctaaggactttgaagatctctttaagcaagaggagtctctgaagaatataaaagatagtctacaacaaagctcaggtcggattgacattattcatagcaagaagacagcagcattgcaaagtgcaacgcctgtggaaagggtgaagctacaggaagctctctcccagcttgatttccaatgggaaaaagttaacaaaatgtacaaggaccgacaagggcgatttgacagatctgttgagaaatggcggcgttttcattatgatataaagatatttaatcagtggctaacagaagctgaacagtttctcagaaagacacaaattcctgagaattgggaacatgctaaatacaaatggtatcttaaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggctagaagaacaaaagaatatcttgtcagaatttcaaagagatttaaatgaatttgttttatggttggaggaagcagataacattgctagtatcccacttgaacctggaaaagagcagcaactaaaagaaaagcttgagcaagtcaagttactggtggaagagttgcccctgcgccagggaattctcaaacaattaaatgaaactggaggacccgtgcttgtaagtgctcccataagcccagaagagcaagataaacttgaaaataagctcaagcagacaaatctccagtggataaaggtttccagagctttacctgagaaacaaggagaaattgaagctcaaataaaagaccttgggcagcttgaaaaaaagcttgaagaccttgaagagcagttaaatcatctgctgctgtggttatctcctattaggaatcagttggaaatttataaccaaccaaaccaagaaggaccatttgacgttcaggaaactgaaatagcagttcaagctaaacaaccggatgtggaagagattttgtctaaagggcagcatttgtacaaggaaaaaccagccactcagccagtgaagaggaagttagaagatctgagctctgagtggaaggcggtaaaccgtttacttcaagagctgagggcaaagcagcctgacctagctcctggactgaccactattggagcctctcctactcagactgttactctggtgacacaacctgtggttactaaggaaactgccatctccaaactagaaatgccatcttccttgatgttggaggtacctgctctggcagatttcaaccgggcttggacagaacttaccgactggctttctctgcttgatcaagttataaaatcacagagggtgatggtgggtgaccttgaggatatcaacgagatgatcatcaagcagaaggcaacaatgcaggatttggaacagaggcgtccccagttggaagaactcattaccgctgcccaaaatttgaaaaacaagaccagcaatcaagaggctagaacaatcattacggatcgaattgaaagaattcagaatcagtgggatgaagtacaagaacaccttcagaaccggaggcaacagttgaatgaaatgttaaaggattcaacacaatggctggaagctaaggaagaagctgagcaggtcttaggacaggccagagccaagcttgagtcatggaaggagggtccctatacagtagatgcaatccaaaagaaaatcacagaaaccaagcagttggccaaagacctccgccagtggcagacaaatgtagatgtggcaaatgacttggccctgaaacttctccgggattattctgcagatgataccagaaaagtccacatgataacagagaatatcaatgcctcttggagaagcattcataaaagggtgagtgagcgagaggctgctttggaagaaactcatagattactgcaacagttccccctggacctggaaaagtttcttgcctggcttacagaagctgaaacaactgccaatgtcctacaggatgctacccgtaaggaaaggctcctagaagactccaagggagtaaaagagctgatgaaacaatggcaagacctccaaggtgaaattgaagctcacacagatgtttatcacaacctggatgaaaacagccaaaaaatcctgagatccctggaaggttccgatgatgcagtcctgttacaaagacgtttggataacatgaacttcaagtggagtgaacttcggaaaaagtctctcaacattaggtcccatttggaagccagttctgaccagtggaagcgtctgcacctttctctgcaggaacttctggtgtggctacagctgaaagatgatgaattaagccggcaggcacctattggaggcgactttccagcagttcagaagcagaacgatgtacatagggccttcaagagggaattgaaaactaaagaacctgtaatcatgagtactcttgagactgtacgaatatttctgacagagcagcctttggaaggactagagaaactctaccaggagcccagagagctgcctcgcctttggaaggactagagactgaggagagagcccagaatgtcactcggcttctacgaaagcaggctgaggaggtcaatactgagtgggaaaaattgaacctgcactccgctgactggcagagaaaaatagatgagacccttgaaagactccaggaacttcaagaggccacggatgagctggacctcaagctgcgccaagctgaggtgatcaagggatcctggcagcccgtgggcgatctcctcattgactctctccaagatcacctcgagaaagtcaaggcacttcgaggagaaattgcgcctctgaaagagaacgtgagccacgtcaatgaccttgctcgccagcttaccactttgggcattcagctctcaccgtataacctcagcactctggaagacctgaacaccagatggaagcttctgcaggtggccgtcgaggaccgagtcaggcagctgcatgaagcccacagggactttggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaacgcctgcctcgtcccctcagctttcacacgatgatactcattcacgcattgaacattatgctagcaggctagcagaaatggaaaacagcaatggatcttatctaaatgatagcatctctcctaatgagagcatagatgatgaacatttgttaatccagcattactgccaaagtttgaaccaggactcccccctgagccagcctcgtagtcctgcccagatcttgatttccttagagagtgaggaaagaggggagcttgagagaatcctagcagatcttgaggaagaaaacaggaatctgcaagcagaatatgaccgtctaaagcagcagcacgaacataaaggcctgtccccactgccgtcccctcctgaaatgatgcccacctctccccagagtccccgggatgctgagctcattgctgaggccaagctactgcgtcaacacaaaggccgcctggaagccaggatgcaaatcctggaagaccacaataaacagctggagtcacagttacacaggctaaggcagctgctggagcaaccccaggcagaggccaaagtgaatggcacaacggtgtcctctccttctacctctctacagaggtccgacagcagtcagcctatgctgctccgagtggttggcagtcaaacttcggactccatgggtgaggaagatcttctcagtcctccccaggacacaagcacagggttagaggaggtgatggagcaactcaacaactccttccctagttcaagaggaagaaatacccctggaaagccaatgagagaggacacaatgtaggaagtcttttccacatggcagatgatttgggcagagcgatggagtccttagtatcagtcatgacagatgaagaaggagcagaataaatgttttacaactcctgattcccgcatggtttttataatattcatacaacaaagaggattagacagtaagagtttacaagaaataaatctatatttttgtgaagggtagtggtattatactgtagatttcagtagtttctaagtctgttattgttttgttaacaatggcaggttttacacgtgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagcggccgc

SEQ ID NO: 10 - DNA, 7112 bp, Artificial
  Mouse promoter and enhancer sequences together with human dystrophin coding sequence
atgagtgccaggaagctgcgaaatctgtcttacaaaaaggtgattgtggaagagtctagaatcttcatttattgttcagcaggattacagaaaagctatcaagagtaaacatttaactgatacactcttattccttctttttaggctgtaaggaggcaaaagttgcttgaacagagcatccagtctgcgcaggagactgaaaaatccttacacttaatccaggagtccctcacattcattgacaagcagttggcagcttatattgcagacaaggtggacgcagctcaaatgcctcaggaagcccagaaaatccaatctgatttgacaagtcatgagatcagtttagaagaaatgaagaaacataatcaggggaaggaggctgcccaaagagtcctgtctcagattgatgttgcacagaaaaaattacaagatgtctccatgaagtttcgattattccagaaaccagccaattttgagcagcgtctacaagaaagtaagatgattttagatgaagtgaagatgcacttgcctgcattggaaacaaagagtgtggaacaggaagtagtacagtcacagctaaatcattgtgtgaacttgtataaaagtctgagtgaagtgaagtctgaagtggaaatggtgataaagactggacgtcagattgtacagaaaaagcagacggaaaatcccaaagaacttgatgaaagagtaacagctttgaaattgcattataatgagctgggagcaaaggtaacagaaagaaagcaacagttggagaaatgcttgaaattgtcccgtaagatgcgaaaggaaatgaatgtcttgacagaatggctggcagctacagatatggaattgacaaagagatcagcagttgaaggaatgcctagtaatttggattctgaagttgcctggggaaaggctactcaaaaagagattgagaaacagaaggtgcacctgaagagtatcacagaggtaggagaggccttgaaaacagttttgggcaagaaggagacgttggtggaagataaactcagtcttctgaatagtaactggatagctgtcacctcccgagcagaagagtggttaaatcttttgttggaataccagaaacacatggaaacttttgaccagaatgtggaccacatcacaaagtggatcattcaggctgacacacttttggatgaatcagagaaaaagaaaccccagcaaaaagaagacgtgcttaagcgtttaaaggcagaactgaatgacatacgcccaaaggtggactctacacgtgaccaagcagcaaacttgatggcaaaccgcggtgaccactgcaggaaattagtagagccccaaatctcagagctcaaccatcgatttgcagccatttcacacagaattaagactggaaaggcctccattcctttgaaggaattggagcagtttaactcagatatacaaaaattgcttgaaccactggaggctgaaattcagcagggggtgaatctgaaagaggaagacttcaataaagatatgaatgaagacaatgagggtactgtaaaagaattgttgcaaagaggagacaacttacaacaaagaatcacagatgagagaaagagagaggaaataaagataaaacagcagctgttacagacaaaacataatgctctcaaggatttgaggtctcaaagaagaaaaaaggctctagaaatttctcatcagtggtatcagtacaagaggcaggctgatgatctcctgaaatgcttggatgacattgaaaaaaaattagccagcctacctgagcccagagatgaaaggaaaataaaggaaattgatcgggaattgcagaagaagaaagaggagctgaatgcagtgcgtaggcaagctgagggcttgtctgaggatggggccgcaatggcagtggagccaactcagatccagctcagcaagcgctggcgggaaattgagagcaaatttgctcagtttcgaagactcaactttgcacaaattcacactgtccgtgaagaaacgatgatggtgatgactgaagacatgcctttggaaatttcttatgtgccttctacttatttgactgaaatcactcatgtctcacaagccctattagaagtggaacaacttctcaatgctcctgacctctgtgctaaggactttgaagatctctttaagcaagaggagtctctgaagaatataaaagatagtctacaacaaagctcaggtcggattgacattattcatagcaagaagacagcagcattgcaaagtgcaacgcctgtggaaagggtgaagctacaggaagctctctcccagcttgatttccaatgggaaaaagttaacaaaatgtacaaggaccgacaagggcgatttgacagatctgttgagaaatggcggcgttttcattatgatataaagatatttaatcagtggctaacagaagctgaacagtttctcagaaagacacaaattcctgagaattgggaacatgctaaatacaaatggtatcttaaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggctagaagaacaaaagaatatcttgtcagaatttcaaagagatttaaatgaatttgttttatggttggaggaagcagataacattgctagtatcccacttgaacctggaaaagagcagcaactaaaagaaaagcttgagcaagtcaagttactggtggaagagttgcccctgcgccagggaattctcaaacaattaaatgaaactggaggacccgtgcttgtaagtgctcccataagcccagaagagcaagataaacttgaaaataagctcaagcagacaaatctccagtggataaaggtttccagagctttacctgagaaacaaggagaaattgaagctcaaataaaagaccttgggcagcttgaaaaaaagcttgaagaccttgaagagcagttaaatcatctgctgctgtggttatctcctattaggaatcagttggaaatttataaccaaccaaaccaagaaggaccatttgacgttcaggaaactgaaatagcagttcaagctaaacaaccggatgtggaagagattttgtctaaagggcagcatttgtacaaggaaaaaccagccactcagccagtgaagaggaagttagaagatctgagctctgagtggaaggcggtaaaccgtttacttcaagagctgagggcaaagcagcctgacctagctcctggactgaccactattggagcctctcctactcagactgttactctggtgacacaacctgtggttactaaggaaactgccatctccaaactagaaatgccatcttccttgatgttggaggtacctgctctggcagatttcaaccgggcttggacagaacttaccgactggctttctctgcttgatcaagttataaaatcacagagggtgatggtgggtgaccttgaggatatcaacgagatgatcatcaagcagaaggcaacaatgcaggatttggaacagaggcgtccccagttggaagaactcattaccgctgcccaaaatttgaaaaacaagaccagcaatcaagaggctagaacaatcattacggatcgaattgaaagaattcagaatcagtgggatgaagtacaagaacaccttcagaaccggaggcaacagttgaatgaaatgttaaaggattcaacacaatggctggaagctaaggaagaagctgagcaggtcttaggacaggccagagccaagcttgagtcatggaaggagggtccctatacagtagatgcaatccaaaagaaaatcacagaaaccaagcagttggccaaagacctccgccagtggcagacaaatgtagatgtggcaaatgacttggccctgaaacttctccgggattattctgcagatgataccagaaaagtccacatgataacagagaatatcaatgcctcttggagaagcattcataaaagggtgagtgagcgagaggctgctttggaagaaactcatagattactgcaacagttccccctggacctggaaaagtttcttgcctggcttacagaagctgaaacaactgccaatgtcctacaggatgctacccgtaaggaaaggctcctagaagactccaagggagtaaaagagctgatgaaacaatggcaagacctccaaggtgaaattgaagctcacacagatgtttatcacaacctggatgaaaacagccaaaaaatcctgagatccctggaaggttccgatgatgcagtcctgttacaaagacgtttggataacatgaacttcaagtggagtgaacttcggaaaaagtctctcaacattaggtcccatttggaagccagttctgaccagtggaagcgtctgcacctttctctgcaggaacttctggtgtggctacagctgaaagatgatgaattaagccggcaggcacctattggaggcgactttccagcagttcagaagcagaacgatgtacatagggccttcaagagggaattgaaaactaaagaacctgtaatcatgagtactcttgagactgtacgaatatttctgacagagcagcctttggaaggactagagaaactctaccaggagcccagagagctgcctcgcctttggaaggactagagactgaggagagagcccagaatgtcactcggcttctacgaaagcaggctgaggaggtcaatactgagtgggaaaaattgaacctgcactccgctgactggcagagaaaaatagatgagacccttgaaagactccaggaacttcaagaggccacggatgagctggacctcaagctgcgccaagctgaggtgatcaagggatcctggcagcccgtgggcgatctcctcattgactctctccaagatcacctcgagaaagtcaaggcacttcgaggagaaattgcgcctctgaaagagaacgtgagccacgtcaatgaccttgctcgccagcttaccactttgggcattcagctctcaccgtataacctcagcactctggaagacctgaacaccagatggaagcttctgcaggtggccgtcgaggaccgagtcaggcagctgcatgaagcccacagggactttggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaacgcctgcctcgtcccctcagctttcacacgatgatactcattcacgcattgaacattatgctagcaggctagcagaaatggaaaacagcaatggatcttatctaaatgatagcatctctcctaatgagagcatagatgatgaacatttgttaatccagcattactgccaaagtttgaaccaggactcccccctgagccagcctcgtagtcctgcccagatcttgatttccttagagagtgaggaaagaggggagcttgagagaatcctagcagatcttgaggaagaaaacaggaatctgcaagcagaatatgaccgtctaaagcagcagcacgaacataaaggcctgtccccactgccgtcccctcctgaaatgatgcccacctctccccagagtccccgggatgctgagctcattgctgaggccaagctactgcgtcaacacaaaggccgcctggaagccaggatgcaaatcctggaagaccacaataaacagctggagtcacagttacacaggctaaggcagctgctggagcaaccccaggcagaggccaaagtgaatggcacaacggtgtcctctccttctacctctctacagaggtccgacagcagtcagcctatgctgctccgagtggttggcagtcaaacttcggactccatgggtgaggaagatcttctcagtcctccccaggacacaagcacagggttagaggaggtgatggagcaactcaacaactccttccctagttcaagaggaagaaatacccctggaaagccaatgagagaggacacaatgtag

SEQ ID NO: 11 - DNA, 40 bp, Homo sapiens
tcccgttactctgatcaacttctggccagtagattcttgc

SEQ ID NO: 12 - DNA, 6968 bp, Homo sapiens
gctgtaaggaggcaaaagttgcttgaacagagcatccagtctgcgcaggagactgaaaaatccttacacttaatccaggagtccctcacattcattgacaagcagttggcagcttatattgcagacaaggtggacgcagctcaaatgcctcaggaagcccagaaaatccaatctgatttgacaagtcatgagatcagtttagaagaaatgaagaaacataatcaggggaaggaggctgcccaaagagtcctgtctcagattgatgttgcacagaaaaaattacaagatgtctccatgaagtttcgattattccagaaaccagccaattttgagcagcgtctacaagaaagtaagatgattttagatgaagtgaagatgcacttgcctgcattggaaacaaagagtgtggaacaggaagtagtacagtcacagctaaatcattgtgtgaacttgtataaaagtctgagtgaagtgaagtctgaagtggaaatggtgataaagactggacgtcagattgtacagaaaaagcagacggaaaatcccaaagaacttgatgaaagagtaacagctttgaaattgcattataatgagctgggagcaaaggtaacagaaagaaagcaacagttggagaaatgcttgaaattgtcccgtaagatgcgaaaggaaatgaatgtcttgacagaatggctggcagctacagatatggaattgacaaagagatcagcagttgaaggaatgcctagtaatttggattctgaagttgcctggggaaaggctactcaaaaagagattgagaaacagaaggtgcacctgaagagtatcacagaggtaggagaggccttgaaaacagttttgggcaagaaggagacgttggtggaagataaactcagtcttctgaatagtaactggatagctgtcacctcccgagcagaagagtggttaaatcttttgttggaataccagaaacacatggaaacttttgaccagaatgtggaccacatcacaaagtggatcattcaggctgacacacttttggatgaatcagagaaaaagaaaccccagcaaaaagaagacgtgcttaagcgtttaaaggcagaactgaatgacatacgcccaaaggtggactctacacgtgaccaagcagcaaacttgatggcaaaccgcggtgaccactgcaggaaattagtagagccccaaatctcagagctcaaccatcgatttgcagccatttcacacagaattaagactggaaaggcctccattcctttgaaggaattggagcagtttaactcagatatacaaaaattgcttgaaccactggaggctgaaattcagcagggggtgaatctgaaagaggaagacttcaataaagatatgaatgaagacaatgagggtactgtaaaagaattgttgcaaagaggagacaacttacaacaaagaatcacagatgagagaaagagagaggaaataaagataaaacagcagctgttacagacaaaacataatgctctcaaggatttgaggtctcaaagaagaaaaaaggctctagaaatttctcatcagtggtatcagtacaagaggcaggctgatgatctcctgaaatgcttggatgacattgaaaaaaaattagccagcctacctgagcccagagatgaaaggaaaataaaggaaattgatcgggaattgcagaagaagaaagaggagctgaatgcagtgcgtaggcaagctgagggcttgtctgaggatggggccgcaatggcagtggagccaactcagatccagctcagcaagcgctggcgggaaattgagagcaaatttgctcagtttcgaagactcaactttgcacaaattcacactgtccgtgaagaaacgatgatggtgatgactgaagacatgcctttggaaatttcttatgtgccttctacttatttgactgaaatcactcatgtctcacaagccctattagaagtggaacaacttctcaatgctcctgacctctgtgctaaggactttgaagatctctttaagcaagaggagtctctgaagaatataaaagatagtctacaacaaagctcaggtcggattgacattattcatagcaagaagacagcagcattgcaaagtgcaacgcctgtggaaagggtgaagctacaggaagctctctcccagcttgatttccaatgggaaaaagttaacaaaatgtacaaggaccgacaagggcgatttgacagatctgttgagaaatggcggcgttttcattatgatataaagatatttaatcagtggctaacagaagctgaacagtttctcagaaagacacaaattcctgagaattgggaacatgctaaatacaaatggtatcttaaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggctagaagaacaaaagaatatcttgtcagaatttcaaagagatttaaatgaatttgttttatggttggaggaagcagataacattgctagtatcccacttgaacctggaaaagagcagcaactaaaagaaaagcttgagcaagtcaagttactggtggaagagttgcccctgcgccagggaattctcaaacaattaaatgaaactggaggacccgtgcttgtaagtgctcccataagcccagaagagcaagataaacttgaaaataagctcaagcagacaaatctccagtggataaaggtttccagagctttacctgagaaacaaggagaaattgaagctcaaataaaagaccttgggcagcttgaaaaaaagcttgaagaccttgaagagcagttaaatcatctgctgctgtggttatctcctattaggaatcagttggaaatttataaccaaccaaaccaagaaggaccatttgacgttcaggaaactgaaatagcagttcaagctaaacaaccggatgtggaagagattttgtctaaagggcagcatttgtacaaggaaaaaccagccactcagccagtgaagaggaagttagaagatctgagctctgagtggaaggcggtaaaccgtttacttcaagagctgagggcaaagcagcctgacctagctcctggactgaccactattggagcctctcctactcagactgttactctggtgacacaacctgtggttactaaggaaactgccatctccaaactagaaatgccatcttccttgatgttggaggtacctgctctggcagatttcaaccgggcttggacagaacttaccgactggctttctctgcttgatcaagttataaaatcacagagggtgatggtgggtgaccttgaggatatcaacgagatgatcatcaagcagaaggcaacaatgcaggatttggaacagaggcgtccccagttggaagaactcattaccgctgcccaaaatttgaaaaacaagaccagcaatcaagaggctagaacaatcattacggatcgaattgaaagaattcagaatcagtgggatgaagtacaagaacaccttcagaaccggaggcaacagttgaatgaaatgttaaaggattcaacacaatggctggaagctaaggaagaagctgagcaggtcttaggacaggccagagccaagcttgagtcatggaaggagggtccctatacagtagatgcaatccaaaagaaaatcacagaaaccaagcagttggccaaagacctccgccagtggcagacaaatgtagatgtggcaaatgacttggccctgaaacttctccgggattattctgcagatgataccagaaaagtccacatgataacagagaatatcaatgcctcttggagaagcattcataaaagggtgagtgagcgagaggctgctttggaagaaactcatagattactgcaacagttccccctggacctggaaaagtttcttgcctggcttacagaagctgaaacaactgccaatgtcctacaggatgctacccgtaaggaaaggctcctagaagactccaagggagtaaaagagctgatgaaacaatggcaagacctccaaggtgaaattgaagctcacacagatgtttatcacaacctggatgaaaacagccaaaaaatcctgagatccctggaaggttccgatgatgcagtcctgttacaaagacgtttggataacatgaacttcaagtggagtgaacttcggaaaaagtctctcaacattaggtcccatttggaagccagttctgaccagtggaagcgtctgcacctttctctgcaggaacttctggtgtggctacagctgaaagatgatgaattaagccggcaggcacctattggaggcgactttccagcagttcagaagcagaacgatgtacatagggccttcaagagggaattgaaaactaaagaacctgtaatcatgagtactcttgagactgtacgaatatttctgacagagcagcctttggaaggactagagaaactctaccaggagcccagagagctgcctcgcctttggaaggactagagactgaggagagagcccagaatgtcactcggcttctacgaaagcaggctgaggaggtcaatactgagtgggaaaaattgaacctgcactccgctgactggcagagaaaaatagatgagacccttgaaagactccaggaacttcaagaggccacggatgagctggacctcaagctgcgccaagctgaggtgatcaagggatcctggcagcccgtgggcgatctcctcattgactctctccaagatcacctcgagaaagtcaaggcacttcgaggagaaattgcgcctctgaaagagaacgtgagccacgtcaatgaccttgctcgccagcttaccactttgggcattcagctctcaccgtataacctcagcactctggaagacctgaacaccagatggaagcttctgcaggtggccgtcgaggaccgagtcaggcagctgcatgaagcccacagggactttggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaacgcctgcctcgtcccctcagctttcacacgatgatactcattcacgcattgaacattatgctagcaggctagcagaaatggaaaacagcaatggatcttatctaaatgatagcatctctcctaatgagagcatagatgatgaacatttgttaatccagcattactgccaaagtttgaaccaggactcccccctgagccagcctcgtagtcctgcccagatcttgatttccttagagagtgaggaaagaggggagcttgagagaatcctagcagatcttgaggaagaaaacaggaatctgcaagcagaatatgaccgtctaaagcagcagcacgaacataaaggcctgtccccactgccgtcccctcctgaaatgatgcccacctctccccagagtccccgggatgctgagctcattgctgaggccaagctactgcgtcaacacaaaggccgcctggaagccaggatgcaaatcctggaagaccacaataaacagctggagtcacagttacacaggctaaggcagctgctggagcaaccccaggcagaggccaaagtgaatggcacaacggtgtcctctccttctacctctctacagaggtccgacagcagtcagcctatgctgctccgagtggttggcagtcaaacttcggactccatgggtgaggaagatcttctcagtcctccccaggacacaagcacagggttagaggaggtgatggagcaactcaacaactccttccctagttcaagaggaagaaatacccctggaaagccaatgagagaggacacaatgtag

SEQ ID NO: 13 - protein, 2329 aa, Homo sapiens
MSARKLRNLSYKKNAVRRQKLLEQSIQSAQETEKSLHLIQESLTFIDKQLAAYIADKVDAAQMPQEAQKIQSDLTSHEISLEEMKKHNQGKEAAQRVLSQIDVAQKKLQDVSMKFRLFQKPANFEQRLQESKMILDEVKMHLPALETKSVEQEVVQSQLNHCVNLYKSLSEVKSEVEMVIKTGRQIVQKKQTENPKELDERVTALKLHYNELGAKVTERKQQLEKCLKLSRKMRKEMNVLTEWLAATDMELTKRSAVEGMPSNLDSEVAWGKATQKEIEKQKVHLKSITEVGEALKTVLGKKETLVEDKLSLLNSNWIAVTSRAEEWLNLLLEYQKHMETFDQNVDHITKWIIQADTLLDESEKKKPQQKEDVLKRLKAELNDIRPKVDSTRDQAANLMANRGDHCRKLVEPQISELNHRFAAISHRIKTGKASIPLKELEQFNSDIQKLLEPLEAEIQQGVNLKEEDFNKDMNEDNEGTVKELLQRGDNLQQRITDERKREEIKIKQQLLQTKHNALKDLRSQRRKKALEISHQWYQYKRQADDLLKCLDDIEKKLASLPEPRDERKIKEIDRELQKKKEELNAVRRQAEGLSEDGAAMAVEPTQIQLSKRWREIESKFAQFRRLNFAQIHTVREETMMVMTEDMPLEISYVPSTYLTEITHVSQALLEVEQLLNAPDLCAKDFEDLFKQEESLKNIKDSLQQSSGRIDIIHSKKTAALQSATPVERVKLQEALSQLDFQWEKVNKMYKDRQGRFDRSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEEQKNILSEFQRDLNEFVLWLEEADNIASIPLEPGKEQQLKEKLEQVKLLVEELPLRQGILKQLNETGGPVLVSAPISPEEQDKLENKLKQTNLQWIKVSRALPEKQGEIEAQIKDLGQLEKKLEDLEEQLNHLLLWLSPIRNQLEIYNQPNQEGPFDVQETEIAVQAKQPDVEEILSKGQHLYKEKPATQPVKRKLEDLSSEWKAVNRLLQELRAKQPDLAPGLTTIGASPTQTVTLVTQPVVTKETAISKLEMPSSLMLEVPALADFNRAWTELTDWLSLLDQVIKSQRVMVGDLEDINEMIIKQKATMQDLEQRRPQLEELITAAQNLKNKTSNQEARTIITDRIERIQNQWDEVQEHLQNRRQQLNEMLKDSTQWLEAKEEAEQVLGQARAKLESWKEGPYTVDAIQKKITETKQLAKDLRQWQTNVDVANDLALKLLRDYSADDTRKVHMITENINASWRSIHKRVSEREAALEETHRLLQQFPLDLEKFLAWLTEAETTANVLQDATRKERLLEDSKGVKELMKQWQDLQGEIEAHTDVYHNLDENSQKILRSLEGSDDAVLLQRRLDNMNFKWSELRKKSLNIRSHLEASSDQWKRLHLSLQELLVWLQLKDDELSRQAPIGGDFPAVQKQNDVHRAFKRELKTKEPVIMSTLETVRIFLTEQPLEGLEKLYQEPRELPPEERAQNVTRLLRKQAEEVNTEWEKLNLHSADWQRKIDETLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETPASSPQLSHDDTHSRIEHYASRLAEMENSNGSYLNDSISPNESIDDEHLLIQHYCQSLNQDSPLSQPRSPAQILISLESEERGELERILADLEEENRNLQAEYDRLKQQHEHKGLSPLPSPPEMMPTSPQSPRDAELIAEAKLLRQHKGRLEARMQILEDHNKQLESQLHRLRQLLEQPQAEAKVNGTTVSSPSTSLQRSDSSQPMLLRVVGSQTSDSMGEEDLLSPPQDTSTGLEEVMEQLNNSFPSSRGRNTPGKPMREDTM

SEQ ID NO: 14 - DNA, 30 bp, Murinae gen. sp.
  misc_feature (1)..(14): This is an MCK-specific forward primer
cccagcaactgagttcttaagtctgaaacc

SEQ ID NO: 15 - DNA, 21 bp, Homo sapiens
  misc_feature (1)..(21): This is a human dystrophin exon 30 specific reverse primer
ctgggcttcctgaggcatttg

SEQ ID NO: 16 - DNA, 368 bp, Artificial
  Amplified Product of mouse creatine kinase specific forward primer and dystrophin human exon 30-specific reverse primer
cccagcaactgagttcttaagtctgaaccctttcttctcacagggtcccaaaggccgccaatatgagtgccaggaagctgcgaaatctgtcttacaaaaaggtgattgtggaagagtctagaatcttcatttattgttcagcaggattacagaaaagctatcaagagtaaacatttaactgatacactcttattccttctttttaggctgtaaggaggcaaaagttgcttgaacagagcatccagtctgcgcaggagactgaaaaatccttacacttaatccaggagtccctcacattcattgacaagcagttggcagcttatattgcagacaaggtggacgcagctcaaatgcctcaggaagcccag

SEQ ID NO: 17 - DNA, 312 bp, Artificial
  Amplification product from neomycin resistance gene primer and the primer from human utrophin exon 64
cccactttgtggttctaagtacgtggtttcaaatgtgtcagtttcatagcctgaagaacgagatcagcagcctctgttccacatacacttcattctcagtattgttttgccaagttctaattccatcagaagctgactctagaggatccgaaggctgcaaaaagcattatgtcgtgagttatcacactaactgaatcaccttgctttgggaggttgactttgttgttatcattaagacagagacaaatggcgaggctgaggtatttgcagagtcaatctgactttcagtgccccctcattgaaggagttagc

SEQ ID NO: 18 - DNA, 25 bp, Escherichia coli
  misc_feature (1)..(25): This sequence is a primer rom the neo gene
cccactttgtggttctaagtactgt

SEQ ID NO: 19 - DNA, 24 bp, Mus musculus
  misc_feature (1)..(24): This is a primer from exon 64 of utrophin.
gctaactccttcaatgagggggca

SEQ ID NO: 20 - DNA, 26 bp, Mus musculus
gaataatgtacgtttctctgcctacc

SEQ ID NO: 21 - DNA, 35 bp, Artificial
  This is the forward primer from the PCDNA 3.1 Hygro plasmid from Invitrogen.
acttatacacgtgcctcgactgtgccttctagttg

SEQ ID NO: 22 - DNA, 42 bp, Artificial
  This is the reverse primer from the PCDNA 3.1 Hygro plasmid from Invitrogen.
ataagaatgcggccgctccccagcatgcctgctattgtcttc

(19) United States
(12) Patent Application Publication
(10) Pub. No.: US 2008/0044393 A1
(43) Pub. Date: Feb. 21, 2008

(54) Retinal dystrophin transgene and methods of use thereof

(76) Inventors: Robert L. White, Shawnee, KS (US); Roger Gaedigk, Shawnee, KS (US); Kathleen Fitzgerald-Gustafson, Kansas City, MO (US)

Correspondence Address: ERICKSON & KLEYPAS, L.L.C., 800 W. 47TH STREET, SUITE 401, KANSAS CITY, MO, 64112, US

(21) Appl. No.: 11/050,911

(22) Filed: Feb. 4, 2005

Related U.S. Application Data
(60) Provisional application No. 60/588,700, filed on Jul. 16, 2004.
(60) Provisional application No. 60/608,252, filed on Sep. 9, 2004.
(60) Provisional application No. 60/613,026, filed on Sep. 24, 2004.

(51) Int. Cl. A61K 31/70; A01K 67/00; A61K 35/00; A61P 43/00; C07H 21/04; C12N 15/00; C12N 5/06

(52) U.S. Cl. 424/93.21; 435/320.1; 435/325; 435/372; 514/44; 536/23.5; 800/13

(57) ABSTRACT

Duchenne muscular dystrophy (DMD) is a progressive muscle disease that is caused by severe defects in the dystrophin gene and results in the patient's death by the third decade. The present invention utilizes the Double Mutant mice (DM) as an appropriate human model for DMD as these mice are deficient for both dystrophin and utrophin (mdx/+, utrn −/−), die at 3 months of age and suffer from severe muscle weakness, pronounced growth retardation, kyphosis, weight loss, slack posture, and immobility. Expression from a transgene of novel human retinal dystrophin Dp260 was shown to prevent premature death and reduce the severe muscular dystrophy phenotype to a mild clinical myopathy. Electromyography, histology, radiography, magnetic resonance imaging, and behavior studies concluded that DM transgenic mice grew normally, had normal spinal curvature and mobility, and had reduced muscle pathology. EMG and histologic data from transgenic DM mice showed decreased abnormalities to levels typical of mild myopathy, while the DM mice exhibited severe abnormalities commonly seen in human dystrophinopathies. The transgenic DM mice also had measurable movement levels comparable to those of untreated mdx mice and controls.

### RELATED APPLICATIONS

The following application claims the benefit of Provisional Application Serial Nos.: 60/588,700; Filed: Jul. 16, 2004; 60/608,252; Filed: Sep. 9, 2004; and 60/613,026; Filed: Sep. 24, 2004, the teachings and contents of which are hereby enclosed by reference.

### SEQUENCE LISTING

The present application contains a sequence listing in both computer readable format and on paper. The computer readable format copies are labeled as 34444.txt Copy 1 and 34444.txt Copy 2. These copies are identical to one another and are identical to the paper copy of the sequence listing included herewith. Each of these sequence listings are expressly incorporated by reference into the present application.

### BACKGROUND OF THE INVENTION

1. Field of the Invention

The present invention relates to Duchenne muscular dystrophy (DMD). More particularly, the present invention is concerned with a novel model for DMD as well as treatments for DMD. Still more particularly, the present invention is concerned with a novel transgene, vectors incorporating this transgene, and methods of incorporating this transgene into animal DNA such that expression of dystrophin occurs. Even more particularly, the present invention relates to in vivo treatment of DMD using the novel transgene.

2. Description of the Prior Art

Duchenne muscular dystrophy (DMD) is the most common neuromuscular disease in boys. It is a recessive X-linked disease characterized by progressive muscle degeneration that leads to severe disability in the second decade of life and fatal cardiac or respiratory failure in the early to mid 20's. Presently there are no treatments that can prolong life or significantly alter the clinical course of the disease. Standard care primarily focuses on maintaining the patients' general health and improving their quality of life. Though glucocorticoids (e.g., prednisolone) have been shown in multiple studies to slow muscle strength decline, their effect is relatively short (18-36 months), and they do not alter the clinical course of the disease.

Mutations in the dystrophin gene result in the absence of dystrophin expression which results in DMD. The 427 kDa isoform of dystrophin links integral membrane proteins to the actin cytoskeleton and is thought to stabilize the sarcolemma during muscle activity. Without dystrophin the membrane loses mechanical stability allowing an influx of calcium ions and ultimately leads to muscle fiber necrosis.

Dystrophin is a multidomain protein consisting of an N-terminal actin-binding domain, a rod domain containing 24 spectrin-like repeats, a cysteine-rich domain, and a C-terminal domain. The two latter domains bind to proteins of the DAP (dystrophin associated protein) complex and the syntrophins. Alternative splicing of the 79 exons of the dystrophin gene produces several dystrophin isoforms, ranging from 71 kDa to the full-length 427 kDa. At least 7 independent promoters drive the transcription of 7 different dystrophin isoforms that are expressed in a cell-specific manner.

The mdx mouse has been used as a genetic model of human DMD. The mdx mice show signs of muscular dystrophy during the first six weeks of life, but unlike DMD in humans, their subsequent disease course is mild. The limb muscles of adult mdx mice do not show the significant weakness or the severe progressive degeneration seen in human DMD. The mdx mouse diaphragm does exhibit degeneration and fibrosis comparable to that in human DMD muscle, but the mice do not suffer respiratory impairment and they have normal lifespans.

Utrophin (utrn) is an autosomal homologue of dystrophin that interacts with the dystrophin-associated proteins and compensates for the lack of muscle dystrophin in mdx mice. Muscles with the maximum upregulation of utrophin exhibit the least pathological changes. However, this compensatory substitution does not occur in humans, which likely explains the phenotypic differences between the mdx mouse and human DMD.

Accordingly, one thing that is needed in the art is a genetic model of human DMD that possesses the same phenotypic characteristics and clinical findings as with human DMD. What is further needed in the art is a gene that expresses dystrophin or a homologue thereof. What is still further needed in the art is a vector that includes a gene that expresses dystrophin, or a homologue thereof, which is capable of transfecting an animal genome such that the dystrophin gene, or homologue thereof, is expressed and thereby compensates for the lack of muscle dystrophin. What is even further needed is a method of treating DMD using cells that have been transfected with DNA expressing dystrophin or a homologue thereof. What is still further needed is a method of treating DMD utilizing the isolated protein expressed by a gene that expresses dystrophin or a homologue thereof. Finally what is needed is a method of treating DMD utilizing a vector wherein the vector transfects the genome of an affected animal and dystrophin or homologue thereof is expressed and compensates for the lack of muscle dystrophin.

### SUMMARY OF THE INVENTION

The present invention overcomes the problems inherent in the prior art and provides a distinct advance in the state of the art. Broadly stated, one aspect of the present invention includes an isolated transgene that contains an isoform of human retinal dystrophin, denominated Dp260, and appropriate regulatory elements. In another aspect of the present invention, methods are provided for incorporating or inserting this Dp260 transgene into a vector for insertion into the genome of an animal, thereby causing it to express retinal dystrophin protein. Preferably, the animal is selected from the group consisting of mammals, more preferably, it is selected from the group consisting of humans, mice, dogs, and horses, and most preferably, the animal is human. In a related aspect of the present invention, the animals containing the Dp260 transgene are provided. In another aspect of the present invention, the Dp260 transgene can be used to transform bone marrow cells and myoblasts for use in gene therapy for muscular dystrophy in animals. Preferably the animals are mammals. More preferably, the animals are selected from the group consisting of mice, dogs, horses, and humans. In another aspect of the present invention, the Dp260 transgene is used in other suitable vectors or with other suitable transfection methods, such as lipofection, for other methods of gene therapy for muscular dystrophy. In another aspect of the present invention, the protein expressed by Dp260 is administered to animals in need thereof.

One embodiment of the present invention is constructed from the DNA sequence of human Dp260. Human Dp260 is an isoform of dystrophin, and is produced by alternative splicing of unique first exon R1 to exon 30 of the dystrophin gene. Human retinal dystrophin contains the cysteine-rich, C-terminal, and most of the rod-like domains found in dystrophin, but lacks dystrophin's N-terminal actin-binding domain. An additional, secondary actin-binding domain has been located in the spectrin repeats of human Dp260. Human Dp260 is normally expressed in the retina, and colocalizes with actin and other dystrophin-related proteins. It may also share many of dystrophin's functions. In this embodiment, a transgene can be constructed from human retinal dystrophin and appropriate regulatory elements. An appropriate human Dp260 sequence may be derived from ATCC clones 57670, 57672, 57674, and 57676, and can be cloned directly into a plasmid through use of techniques known in the art. For purposes of the present invention, preferred DNA sequences for use in a transgene should have the same function as human Dp260, more preferably, the DNA sequence of the Dp260 portion of the transgene should have at least 80%, more preferably at least 85%, still more preferably at least 90%, even more preferably at least 95%, still more preferably at least 97%, and most preferably 99-100% sequence identity with human Dp260. The transgene sequence of the present invention can also be an isoform resulting from alternative splicing of dystrophin. One such alternatively spliced form of dystrophin useful for purposes of the present invention contains dystrophin exon 71. In preferred forms, the final transgene also contains promoter and enhancer sequences upstream of the Dp260 sequence to facilitate expression of the transgene. Preferred regulatory elements include mouse muscle creatine kinase (MCK) promoter and enhancer, and mouse MCK exons 1 and 2 as regulatory elements. Transgene expression is tested by stable transfection of the transgene into a cell line, and subsequent sequencing analysis of the protein product. Errors in splicing are fixed by conventional site-directed mutagenesis to improve the exon acceptor scores of the correct splice sites. In other preferred forms, the transgene contains additional regulatory sites to ensure proper stability of the resulting transcript. One such regulatory site is a bovine growth hormone (BGH) poly A signal sequence added to the 3′ end of the construct to ensure proper polyadenylation.

In another embodiment, the present invention includes the Dp260 transgene and its associated regulatory elements, as described above, in a vector suitable for transfecting other cells. Such a vector preferably contains a DNA sequence which expresses a protein having a function similar to that of dystrophin. Preferably, the DNA sequence used in such a vector will have at least 80%, more preferably at least 85%, still more preferably at least 90%, even more preferably at least 95%, still more preferably at least 97%, and most preferably 99-100% sequence identity with human Dp260. In some preferred forms, the vector also contains a form of human Dp260 that includes human dystrophin exon 71. In more preferred forms, the vector also contains regulatory elements such as promoters, enhancers, and poly A signal sites, as described above. This vector could be a variety of commercially available plasmids, adenoviruses, or lentiviruses.

In another embodiment, the present invention includes an animal transfected with a Dp260 transgene. In preferred forms, the Dp260 used for transfection expresses a protein having similar function to dystrophin, and preferably, the Dp260 is human Dp260. The genome of such an animal should contain at least one copy of a DNA sequence preferably having at least 80%, more preferably at least 85%, still more preferably at least 90%, even more preferably at least 95%, still more preferably at least 97%, and most preferably 99-100% sequence identity with human Dp260. In some forms, the animal has at least one copy of a sequence of Dp260 which includes dystrophin exon 71, located in their genome. Preferably, the animal is a mammal, and more preferably, the animal is selected from the group consisting of humans, mice, horses, and dogs.

In another embodiment of the present invention, a Dp 260 transgene is inserted into an animal's genome by a microinjection process that includes freeing the transgene from its plasmid by restriction digest, and injecting it directly into the animal's oocytes. Animals that have incorporated the transgene into their genome are identified by appropriate conventional methods including sequencing and PCR reactions. Preferably, these animals express Dp260 in their muscle cells, a property that can be tested using conventional techniques such as PCR and western blotting. Animals benefitting from such an embodiment include humans, mice, dogs, and horses. In one example of this embodiment, the preferred human Dp260 transgene was inserted into the genome of double mutant (DM) mice by injecting the Dp260 transgene into DM mouse oocytes, followed by a series of crosses with mdx and utrophin knockout mice. Of course, mice could also be transfected through any conventional method including by the use of other vectors such as adenoviruses or lentiviruses, as well as electoporation of naked DNA. Untransformed DM mice exhibit physiological symptoms similar to muscular dystrophy in humans, and produce neither dystrophin, nor its murine analogue, utrophin. Additionally, DM mice show a severe phenotype, have short lifespans, have high levels of necrosis in their muscles, and exhibit an increasing incidence of Complex Repetitive Discharges (CRDs), a hallmark of muscular dystrophy, as they age. In contrast, DM mice expressing the Dp260 transgene (DM/Tg+) show symptoms of only a mild myopathy, and have normal lifespans. Additionally, DM/Tg+ mice do not have the severe spinal curvature (kyphosis) or limb muscle weakness seen in DM mice. They also show lower levels of necrosis and lower incidence of CRDs as they age. Due to the similarities between DM mice and human individuals that suffer from DMD, the DM mice appear to be an ideal model for the disease.

In yet another embodiment of the invention, the Dp260 transgene is used to stably transfect cells extracted from mice, dogs, horses and humans. This can be performed with the use of lentiviral vectors incorporating a selectable marker (i.e. neomycin resistance). Preferably, the transfected cells are myoblasts, because such cells differentiate into muscle cells. More preferably, the transfected cells are bone marrow cells, even more preferably, the transfected cells will be side population bone marrow cells, and most preferably, the transfected cells will be side population cells with Lin−, Sca+ and Kit+ cell-surface markers. These transfectant cells are identifiable through known methods such as fluorescence-activated cell sorting (FACS). They can further be defined by their ability to exclude Hoechst dye. Additionally, these transfectant cells show an increased likelihood of differentiating into muscle cells. Methods for transforming these cells include the use of vectors such as plasmids, adenoviruses, lentiviruses, and more preferably, electroporation of naked DNA. Stable expression of Dp260 can be detected through the use of PCR and western blotting experiments.

In still another embodiment of the present invention, methods of supplying Dp260 in animals through the use of gene therapy is provided. Preferably, the animals are mammals, and more preferably are selected from the group consisting of humans, mice, dogs, and horses. The goal of such therapy would be the alleviation of muscular dystrophy symptoms. In one preferred form of this embodiment, cells would be removed from the patient, and stably transfected with a transgene preferably containing a DNA sequence having at least 80%, more preferably at least 85%, still more preferably at least 90%, even more preferably at least 95%, still more preferably at least 97%, and most preferably 99-100% sequence identity with human Dp260. In some preferred forms of this method, such cells would be transfected with a DNA sequence containing a form of Dp260 that includes human dystrophin exon 71. Preferred transgenes of the present invention would also include the appropriate regulatory elements for stable expression of Dp260. Preferably, the cells transfected would be myoblast or bone marrow cells. Even more preferably, these cells would be side population bone marrow cells, as described above, with cell surface markers as described above, such cells being particularly likely to differentiate into muscle cells. Most preferably, these cells would be taken from the patient receiving therapy, transfected outside the body with the Dp260 transgene, and replaced in the same patient in an autologous transplant. Such autologous transplantation decreases the likelihood of generating an immune response, and may further eliminate the need for immunosuppression, as the transfected cells are the patient's own. Autologous bone marrow transplants of transfected cells could be used at a variety of points in time in the course of the disease. Bone marrow cells are more strongly attracted to more damaged cells, thus making this procedure appropriate for older patients who have suffered muscular dystrophy for long periods of time. Also, this process could occur several times throughout a patient's lifetime, because the effects of such autologous bone marrow transplants are additive, thereby increasing healthy, functional muscle mass.

Importantly, the present invention is advantageous in an immunological sense. In general terms, an obstacle to any type of gene therapy is the immunogenicity of the transgene product. Full length dystrophin can induce an immunogenic response which can result in failed expression of the transgene (1). The unique nature of the Dp260 transgene is that it expresses a naturally occurring isoform of human dystrophin. The Dp260 protein is expressed primarily in retina and in small amounts in other tissues. Therefore, retinal dystrophin is a natural isoform. The introduction of Dp260 from a transgene will not induce an immunogenic response especially in patients that have deletions upstream of exon 30 which do not affect the expression of Dp260. This is a distinct advantage over full length dystrophin transgenes as well as micro-dystrophin transgenes in which most of the spectrin domain coding region is removed or gutted. The microdystrophins will also potentially induce an immunogenic response since the protein can be considered a neoantigen (the microdystrophin protein contains sequences which are foreign to patients with Duchenne muscular dystrophy). The Dp260 transgene of the present invention overcomes this important barrier to successful gene therapy.

As used herein, the following definitions will apply: “Sequence Identity” as it is known in the art refers to a relationship between two or more polypeptide sequences or two or more polynucleotide sequences, namely a reference sequence and a given sequence to be compared with the reference sequence. Sequence identity is determined by comparing the given sequence to the reference sequence after the sequences have been optimally aligned to produce the highest degree of sequence similarity, as determined by the match between strings of such sequences. Upon such alignment, sequence identity is ascertained on a position-by-position basis, e.g., the sequences are “identical” at a particular position if at that position, the nucleotides or amino acid residues are identical. The total number of such position identities is then divided by the total number of nucleotides or residues in the reference sequence to give % sequence identity. Sequence identity can be readily calculated by known methods, including but not limited to, those described in Computational Molecular Biology, Lesk, A. N., ed., Oxford University Press, New York (1988), Biocomputing: Informatics and Genome Projects, Smith, D. W., ed., Academic Press, New York (1993); Computer Analysis of Sequence Data, Part I, Griffin, A. M., and Griffin, H. G., eds., Humana Press, New Jersey (1994); Sequence Analysis in Molecular Biology, von Heinge, G., Academic Press (1987); Sequence Analysis Primer, Gribskov, M. and Devereux, J., eds., M. Stockton Press, New York (1991); and Carillo, H., and Lipman, D., SIAM J. Applied Math., 48: 1073 (1988), the teachings of which are incorporated herein by reference. Preferred methods to determine the sequence identity are designed to give the largest match between the sequences tested. Methods to determine sequence identity are codified in publicly available computer programs which determine sequence identity between given sequences. Examples of such programs include, but are not limited to, the GCG program package (Devereux, J., et al., Nucleic Acids Research, 12(1):387 (1984)), BLASTP, BLASTN and FASTA (Altschul, S. F. et al., J. Molec. Biol., 215:403-410 (1990). The BLASTX program is publicly available from NCBI and other sources (BLAST Manual, Altschul, S. et al., NCVI NLM NIH Bethesda, Md. 20894, Altschul, S. F. et al., J. Molec. Biol., 215:403-410 (1990), the teachings of which are incorporated herein by reference). These programs optimally align sequences using default gap weights in order to produce the highest level of sequence identity between the given and reference sequences. As an illustration, by a polynucleotide having a nucleotide sequence having at least, for example, 95% “sequence identity” to a reference nucleotide sequence, it is intended that the nucleotide sequence of the given polynucleotide is identical to the reference sequence except that the given polynucleotide sequence may include up to 5 point mutations per each 100 nucleotides of the reference nucleotide sequence. In other words, in a polynucleotide having a nucleotide sequence having at least 95% identity relative to the reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence. Analogously, by a polypeptide having a given amino acid sequence having at least, for example, 95% sequence identity to a reference amino acid sequence, it is intended that the given amino acid sequence of the polypeptide is identical to the reference sequence except that the given polypeptide sequence may include up to 5 amino acid alterations per each 100 amino acids of the reference amino acid sequence. In other words, to obtain a given polypeptide sequence having at least 95% sequence identity with a reference amino acid sequence, up to 5% of the amino acid residues in the reference sequence may be deleted or substituted with another amino acid, or a number of amino acids up to 5% of the total number of amino acid residues in the reference sequence may be inserted into the reference sequence. These alterations of the reference sequence may occur at the amino or the carboxy terminal positions of the reference amino acid sequence or anywhere between those terminal positions, interspersed either individually among residues in the reference sequence or in the one or more contiguous groups within the reference sequence. Preferably, residue positions which are not identical differ by conservative amino acid substitutions. However, conservative substitutions are not included as a match when determining sequence identity. It is also understood that the DNA coding for a particular protein may, due to the degeneracy of the code, differ in nucleotide sequence but still express or code for the same protein. Such minor alterations in DNA coding are well understood by those of skill in the art and are covered in the present invention.

As used herein, the term “transfection” means the introduction of a nucleic acid, e.g., via an expression vector, into a recipient cell by nucleic acid-mediated gene transfer. “Transformation”, as used herein, refers to a process in which a cell's genotype is changed as a result of the cellular uptake of exogenous DNA or RNA, and, for example, the transformed cell expresses a recombinant form of a dystrophin protein, or, in the case of anti-sense expression from the transferred gene, the expression of a naturally-occurring form of the dystrophin protein is disrupted.

As used herein, the term “transgene” means a nucleic acid sequence (encoding, e.g., a dystrophin protein, or an antisense transcript thereto), which is partly or entirely heterologous, i.e., foreign, to the transgenic animal or cell into which it is introduced, or, is homologous to an endogenous gene of the transgenic animal or cell into which it is introduced, but which is designed to be inserted, or is inserted, into the animal's genome in such a way as to alter the genome of the cell into which it is inserted (e.g., it is inserted at a location which differs from that of the natural gene or its insertion results in a knockout). A transgene can include one or more regulatory sequences and any other nucleic acid, such as introns, that may be necessary for optimal expression of a selected nucleic acid.

The term “vector” refers to a nucleic acid molecule capable of transporting another nucleic acid to which it has been linked. Preferred vectors are those capable of autonomous replication and/expression of nucleic acids to which they are linked.

A “transgenic” animal is any animal containing cells that bear genetic information received, directly or indirectly, by deliberate genetic manipulation at the subcellular level, such as by microinjection or infection with recombinant virus through a vector or electroporation of naked DNA. “Transgenic” in the present context does not encompass classical crossbreeding or in vitro fertilization, but rather denotes animals in which one or more cells receive a recombinant DNA molecule. Although it is highly preferred that this molecule be integrated within the animal's chromosomes, the invention also encompasses the use of extrachromosomally replicating DNA sequences, such as might be engineered into yeast artificial chromosomes. Preferably transgenic animals of the present invention include “germ cell line transgenic animals,” which refers to a transgenic animal in which the genetic information has been taken up and incorporated into a germ line cell, therefore conferring the ability to transfer the information to offspring. If such offspring, in fact, possess some or all of that information, then they, too, are transgenic animals.

As used herein, the term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides.

The term “stable transfection” or “stably transfected” refers to the introduction and integration of foreign DNA into the genome of the transfected cell. The term “stable transfectant” refers to a cell which has stably integrated foreign DNA into the genomic DNA.

### BRIEF DESCRIPTION OF THE DRAWINGS

The patent or application file contains at least one drawing or photograph executed in color. Copies of this patent or patent application publication with color drawings(s) or photograph(s) will be provided by the Office upon request and payment of the necessary fee.

FIG. 1 is a schematic diagram of the human Dp260 transgene construct indicating all restriction sites utilized in the construction of the transgene;

FIG. 2a is a western blot gel analysis of myoblasts transfected with the Dp260 transgene construct as compared with myoblasts transfected with the MCK plasmid only;

FIG. 2b is a western blot gel analysis of hindlimb muscles of DM/Tg+ and DM mice;

FIG. 3a is a photograph of a transverse section of soleus muscle from an 8-week-old DM/Tg+ mouse immunolabeled with a monoclonal C-terminal specific anti-dystrophin and detected with Alexa-488 conjugated secondary antibody;

FIG. 3b is a photograph of a transverse section of soleus muscle from an 8-week-old DM mouse immunolabeled with a monoclonal C-terminal specific anti-dystrophin and detected with Alexa-488 conjugated secondary antibody;

FIG. 3c is a photograph of a transverse section of soleus muscle from a sixteen-week-old DM/Tg+ mice immunolabeled with a monoclonal C-terminal specific anti-dystrophin and detected with Alexa-488 conjugated secondary antibody;

FIG. 4a is a photograph comparing of the relative sizes and presentations of DM/Tg+ and DM mice;

FIG. 4b is a radiographic xray image of a DM/Tg+ mouse, wherein spinal curvature was measured by goniometric analysis;

FIG. 4c is a radiographic xray image of a DM mouse, wherein spinal curvature was measured by goniometric analysis;

FIG. 4d is a magnetic resonance imaging (MRI) study of a DM/Tg+ mouse;

FIG. 4e is an MRI study of a DM mouse;

FIG. 4f is an MRI study of a normal control mouse;

FIG. 5a is an electromyography (EMG) trace from a DM/Tg+ mouse;

FIG. 5b is an EMG trace from a DM mouse;

FIG. 5c is a graph of the average number of muscle belly quadrants exhibiting complex repititive discharges (CRDs) as DM and DM/Tg+ mice age;

FIG. 5d is a graph showing the average total number of CRDs as DM and DM/Tg+ mice age;

FIG. 6a is a photograph of a toluidine blue-stained transverse section of the soleus muscle of an eight-week-old DM/Tg+ mouse;

FIG. 6b is a photograph of a toluidine blue-stained transverse section of the soleus muscle of an eight-week-old DM mouse;

FIG. 6c is a photograph of a toluidine blue-stained transverse section of the soleus muscle of an eight-week-old wild type mouse;

FIG. 7a is a graph quantifying the percentage of necrotic area in extensor digitorum longus muscles of DM and DM/Tg+ mice, correlated with age;

FIG. 7b is a graph quantifying the percentage of necrotic area in soleus muscles of DM and DM/Tg+ mice, correlated with age;

FIG. 8a is a graph quantifying the percentage of muscle fibers showing centralized nuclei in the extensor digitorum longus of DM and DM/Tg+ mice, correlated with age;

FIG. 8b is a graph quantifying the percentage of muscle fibers showing centralized nuclei in the soleus muscles of DM and DM/Tg+ mice, correlated with age;

FIG. 9 is a bar graph of the locomotor activity as determined using a force plate actometer of DM, DM/Tg+, adult mdx, and adult C57BL/6J mice, wherein the brackets for each error bar represent ±1 standard error of the mean, and the horizontal dashed lines show the 95% confidence interval for the three locomotor activity sessions experienced by the DM/Tg+ mice; and

FIG. 10 is a depiction of the Dp260 transgene with all restriction sites and regions of interest annotated.

### DETAILED DESCRIPTION OF THE PREFERRED EMBODIMENTS

The following examples set forth preferred methods in accordance with the invention. It is to be understood, however, that these examples are provided by way of illustration and nothing therein should be taken as a limitation upon the overall scope of the invention.

### EXAMPLE 1

### Preparation and Analysis of Human Dp260 Transgene Construct

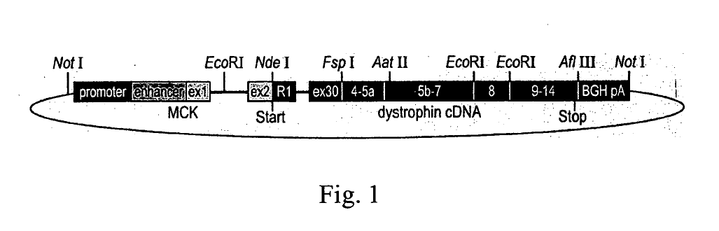

The mouse muscle creatine kinase (MCK) promoter and enhancer (SEQ ID NO: 1), along with MCK exon 1 (SEQ ID NO: 2), intron 1 (SEQ ID NO: 3), and a portion of exon 2 (SEQ ID NO: 4) comprising the 5′ untranslated portion of exon 2, were used to produce the final transgene. The regulatory elements of MCK with its first exon and part of its first intron (SEQ ID NO: 5) were cloned directly into a pBluescript II SK vector (Stratagene, La Jolla, Calif.). The first PCR amplicon, consisting of the remainder of MCK intron 1 and exon 2, up to the MCK ATG start codon (SEQ ID NO: 6), was amplified by PCR to generate an NdeI restriction site. This allowed ligation to the NdeI restriction site of a human genomic PCR amplicon. The second PCR amplicon started with the ATG start codon of the retinal dystrophin unique first exon R1, continued with intron R1, and ended in exon 30 (SEQ ID NO: 7), which was placed at the exact position where the MCK start codon is normally located. The second PCR amplicon (SEQ ID NO: 7) also contained an engineered FspI site. The third PCR product was amplified using the human dystrophin cDNA clone cDMD 4-5a (ATCC No. 57670). This product was designed to contain an FspI restriction site at its 5′ end and a naturally occurring AatII site at its 3′ end, and was added to the construct. The remainder of the human dystrophin coding sequence was created by ligating three human dystrophin cDNA clones, cDMD 5b-7, 8, and 9-14 (ATCC Nos. 57672, 57674, and 57676), to the construct using naturally occurring restriction sites. A bovine growth hormone (BGH) poly A signal sequence (SEQ ID NO: 8) (Invitrogen, Carlsbad, Calif.) was added to the 3′ end of the construct to guarantee proper stability and polyadenylation of the transcript. This signal sequence was generated from a PCR product using the PCDNA 3.1 Hygro plasmid primers from the Invitrogen.com website. The primer sequences are included herein as SEQ ID NOS: 21 and 22, respectively. SEQ ID NO: 21 includes the AflIII restriction site in the BGH-Afl, down primer. SEQ ID NO: 22 includes the NotI restriction site in the BGH-Not, up primer. This yielded the construct shown in FIG. 1 (SEQ ID NO: 9), with all restriction sites used for construction shown.

An ABI 377 automated sequencer (Applied Biosystems, Foster City, Calif.) was used to confirm the sequence accuracy of the entire coding region of the Dp260 transgene (SEQ ID NO: 10). Two silent mutations that retained the wild type amino acid sequence were discovered. Two other changes in the sequence were discovered, and were reverted to wild type sequence by site directed mutagenesis according to the manufacturer's protocol (Quick Change Site Directed Mutagenesis Kit, Stratagene). Sequencing also revealed that the construct lacked exon 71 (SEQ ID NO: 11). This is a result of a normal splice variant in the human and mouse genes, and the syntrophin binding sites are downstream of this exon. Expression of the human Dp260 trangene transcript and protein products was tested by stable transfection in the MM14 myoblast cell line (Hauschka, University of Washington), a line of differentiated muscle cells, according to the methods of Jaynes et al. in Mol. Cell. Biol. 6:2855-2865 (1986), the teachings and content of which is hereby incorporated by reference.

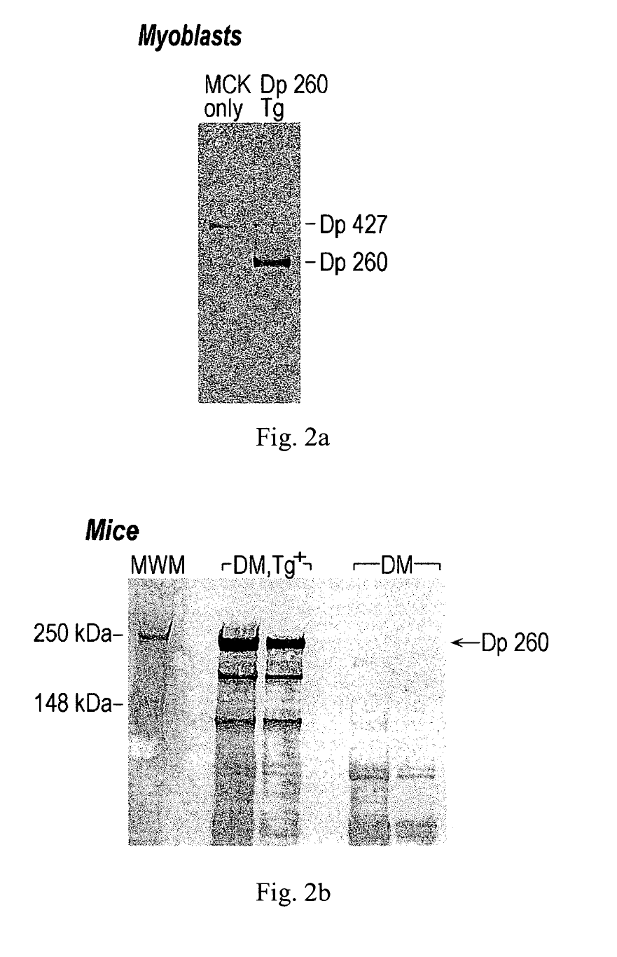

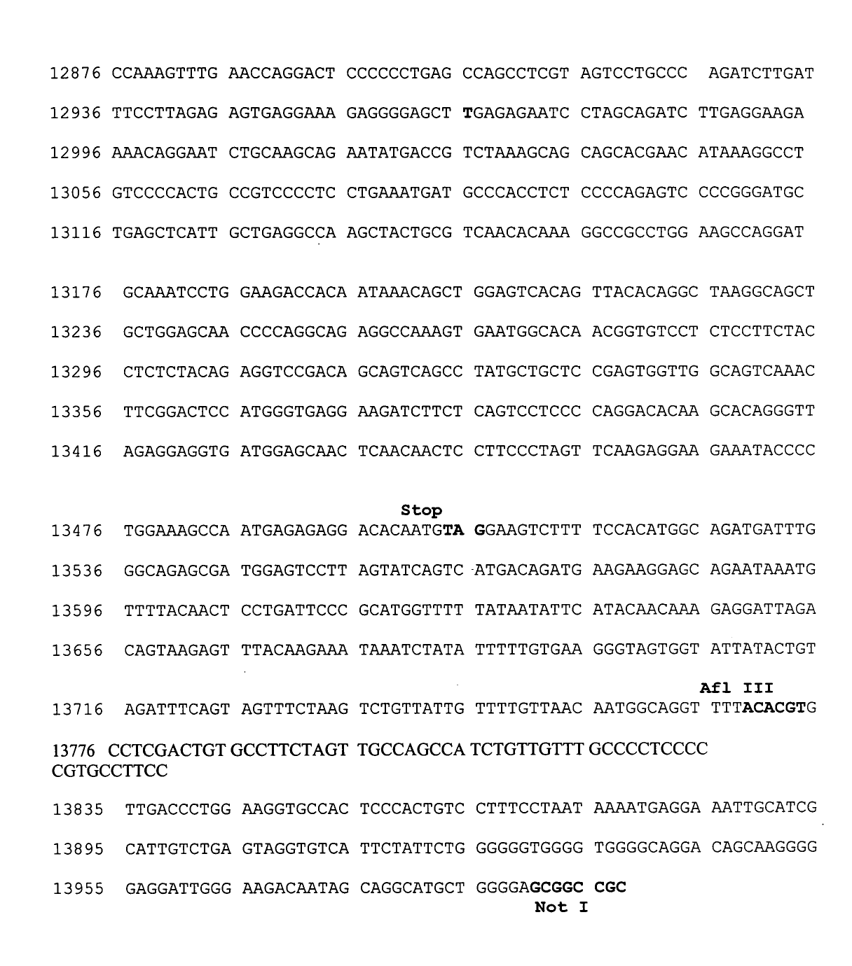

After establishing stable transfection of the transgene into the MM14 myoblast cell line, cDNA PCR product analysis and sequencing showed that most of the transgenic mRNA was spliced from MCK exon 1 directly to dystrophin exon 30, deleting the MCK exon 2/exon R1 segment (SEQ ID NO: 12). Information content analysis showed a strong exon 30 acceptor site score of 12.1 bits compared to a much weaker 6.2 bit score of the MCK exon 2 acceptor. Three nucleotides in the 3′ region of MCK intron 1 were changed by site directed mutagenesis (Quick Change Site Directed Mutagenesis Kit, Stratagene), increasing the bit score for the exon 2 acceptor to 12.4 bits, making it a stronger splice acceptor site. Subsequent transfection experiments confirmed the correct splicing of the RNA product. The mutated nucleotides are found in SEQ ID NO: 5 at positions 6363, 6364, and 6368 (marked with an asterisk in FIG. 10) and all were mutated from “g” to “t.” The expressed protein (SEQ ID NO: 13) was analyzed using western blots of protein preparations made from the transfected myoblasts. The western blots showed robust expression of Dp260 protein in transfected cells, as compared to Dp427 (FIG. 2a). The control transfection using the MCK plasmid without insert showed no expression of Dp260 protein, but did show expression of Dp427 muscle dystrophin.

### EXAMPLE 2

### Production of DM Human Dp260 Transgenic Mice

The human Dp260 transgene construct was extracted with the Endo Free Plasmid Kit (Quiagen, Valencia, Calif.) and was released from the plasmid vector by restriction digest with NotI prior to oocyte injection. The construct was injected into 200 eggs, which were then transplanted into psuedopregnant females, delivered, and weaned. Genotyping for the Dp260 transgene identified two mice that had incorporated the human Dp260 transgene. Genotyping was performed by PCR reactions using an MCK-specific forward primer (SEQ ID NO: 14) and a dystrophin human exon 30-specific reverse primer (SEQ ID NO: 15) which amplified a transgene-specific product of less than 400 bp (SEQ ID NO: 16). Both lines of mice showed strong expression of the transgene and may differ by the location of insertion into the genome, and the number of copies of the transgene inserted into the mouse's genome. The transgenic mice thusly identified as having the TgN(DMD 260)1Raw transgene are henceforth described as Tg+ animals.

Utrophin knockout utrn^{-/-} mice (Stephen Hauschka, University of Washington) were identified using a PCR reaction based on the presence or absence of the inserted neomycin (neo) resistance gene in exon 64 of the utrophin gene. A 312 bp amplicon (SEQ ID NO: 17) was produced using primers developed from sequences of the inserted neo gene (SEQ ID NO: 18) and the 3′ end of exon 64 of the utrophin gene (SEQ ID NO: 19). The wild type allele was identified using an additional forward primer (SEQ ID NO: 20) to the 5′ end, deleted in the utrn knockout mouse. Congenic C57BL/6J lines for the utrn knockout and Tg+ mice were generated by backcrossing to C57BL/6J mice for 10 generations.

The DM (utrn^{-/-}, mdx) males, with and without the transgene, were generated from a series of matings using the utrn knockout mice, the Tg+ mice, and the mdx mice (obtained from The Jackson Laboratory, Bar Harbor, Me.). Mice carrying the mdx mutation were identified using the ARMS PCR assay as previously described by Amalfitano & Chamberlain in Muscle & Nerve 19:1549-1553 (1996). The first mating of mdx females to utrn^{-/-} males produced females which were subsequently mated to Dp260 Tg+ males. This produced female carriers (X^{mdx}, X⁺, utrn^{-/-}, Tg+) which were mated to homozygous utrn^{-/-} males to produce DM males (X^{mdx}Y, utrn^{-/-}) with and without the human Dp260 transgene. These crosses resulted in 48 DM mice, and 48 DM/Tg+ mice.

### EXAMPLE 3

### Western Blotting

Differentiated MM14 myoblast cell cultures, stably transfected with either the human MCK/Dp260 Tg or the MCK plasmid alone, were harvested. Protein was extracted from 3 million cells by homogenizing in 1 mL of homogenization buffer (50 mM Tris pH 8, 150 mM NaCl, 1 mM EDTA, 0.04 mg/mL aprotinin, 0.0025 mg/mL pepstatin A, 0.025 leupeptin, 1 mM phenylmethyl sulfonylfluoride, 0.1% Triton X100) in a Dounce homogenizer. Muscle tissue was also harvested (100 mg) from the hind legs of DM/Tg+, and DM mice. The tissue was frozen and was homogenized in 1 mL homogenization buffer using a chilled mortar and pestle. The homogenates were centrifuged for 10 minutes at 13,000 rpm at 4° C. to sediment cell debris.

A 4× loading buffer (Invitrogen) was added to the supernatant, and the proteins were heat denatured at 70° C. for 10 minutes. Aliquots of 24 μL were analyzed on 4-8% acrylamide gels using a NuPAGE Tris-Acetate SDS Gel System (Invitrogen). Proteins were transferred in a Novex chamber (Invitrogen) to a Hybond-C super membrane (Amersham Biosciences, Piscataway, N.J.). The membrane was blocked overnight at 4° C. in Tris-NaCl-Tween buffer (TNT) with 4% milk to prevent nonspecific binding. Membrane was subsequently incubated for two hours with primary antibody at room temperature. For the myoblast western blots, the primary antibody (VIA4-2 A3, Upstate Biotechnology, Lake Placid, N.Y.) was a mouse monoclonal IgM raised against the last 17 amino acids of the carboxy terminus of dystrophin. For the limb muscle western blots, the primary antibody was a dystrophin C-terminal specific IgG (MANDRA-1, Sigma).

For the myoblast preparation, the membrane underwent several washes using TNT buffer. A secondary antibody (anti-mouse IgM, peroxidase conjugated, Sigma) was applied for 1 hour at room temperature, or overnight at 4° C. After additional washes, the membrane was exposed to an ECL (enhanced chemilluminescence) detection solution (Amersham Biosciences, Piscataway, N.J.) and subsequently exposed to x-ray film. For the hindlimb muscle western blots, an anti-mouse IgG alkaline phosphatase conjugate (Sigma) was used with a BCIP/NBT (5-bromo-4-chloro-3-indolyl-phosphate/nitroblue tetrazolium chloride) kit (KPL, Gaithersburg, Md.) for colorimetric visualization of dystrophin protein bands.

Western blot analysis of mouse hindlimb muscles showed strong expression of Dp260 in DM/Tg+ mice, while western blot analysis of hindlimb muscles from DM mice showed no Dp260 expression.

### EXAMPLE 4

### Immunocytochemistry and Histological Studies

Hind limbs from freshly sacrificed animals were skinned and immersed in 2% paraformaldehyde in phosphate buffered saline, pH 7.4 (PBS), for four to six hours. Soleus and extensor digitorum longus (EDL) muscles from one hind limb were dissected out, fixed for 24 to 48 hours at 4° C., and then embedded in paraffin. They were then sectioned and stained with toluidine blue using standard histological methods. Muscles from the contralateral limb were dissected into 1-2 mm³ blocks, cryoprotected with a mixture of sucrose and polyvinylpyrrolidone according to Tokuyasu in Histochem J. 21:163-171 (1989), and flash frozen in liquid nitrogen. Transverse sections 1.5 μL thick were obtained using a Reichert Ultracut S microtome with an FCS attachment.

Frozen sections were blocked overnight at 4° C. in TBS (50 mM Tris, 150 mM NaCl, 0.001% NaN₃, pH 7.6) containing 0.2% gelatin and 0.5% nonfat dry milk. Sections were washed with TBS for 5 minutes at room temperature, and then incubated for 90 minutes in primary antibody diluted in the blocking solution. Antibodies used were C-terminal specific monoclonal anti-dystrophin (MANDRA-1) diluted 1:25 (Sigma), or rabbit polyclonalantilaminin diluted 1:200 (Sigma). Sections were rinsed for five minutes twice in PBS, blocked for 30 minutes in TBS with 5% goat serum, and rinsed twice with TBS. They were incubated for 60 minutes with an Alexa-488 conjugated, species-specific secondary antibody (Molecular Probes, Eugene, Oreg.), then rinsed and mounted for viewing. Laminin-labeled slides were counterstained with 0.2 mg/mL propidium iodide for 10 minutes to visualize nuclei, then rinsed and mounted again. Images were recorded using an Olympus BX-50 epifluorescence microscope equipped with a CCD camera.

For quantitative analysis of histological sections, cross-sectional areas were digitized on a Macintosh computer using the public domain NIH Image program. Values were expressed as percentages of necrosis/regeneration per total muscle cross-sectional area. Percentages of muscle fibers with non-peripheral nuclei were determined using digital images of frozen sections labeled with propidium iodide and anti-laminin. Differences between means were analyzed using the Student's t-test.

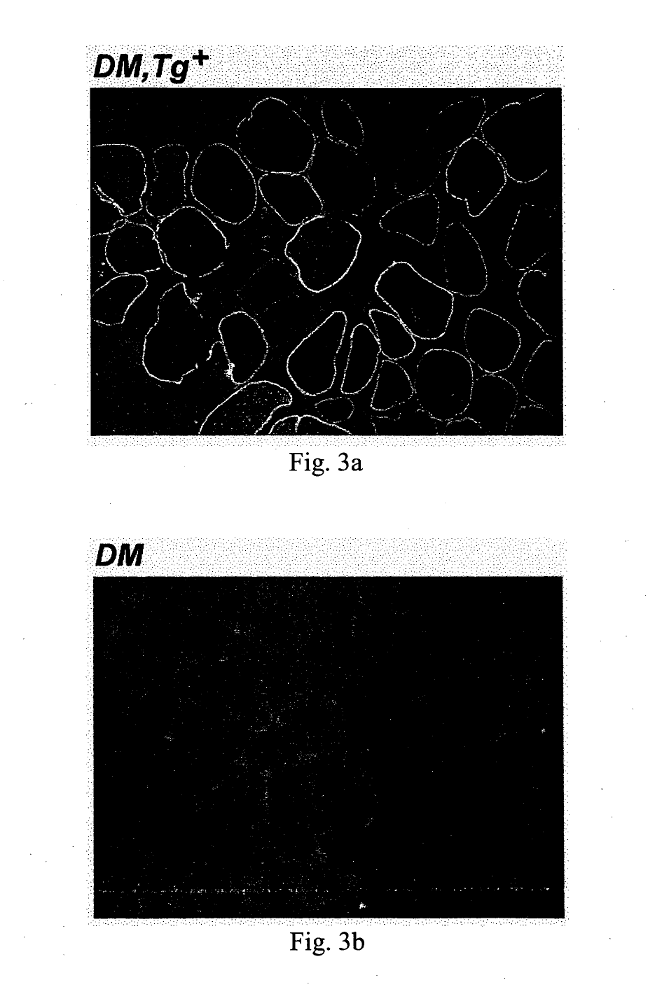

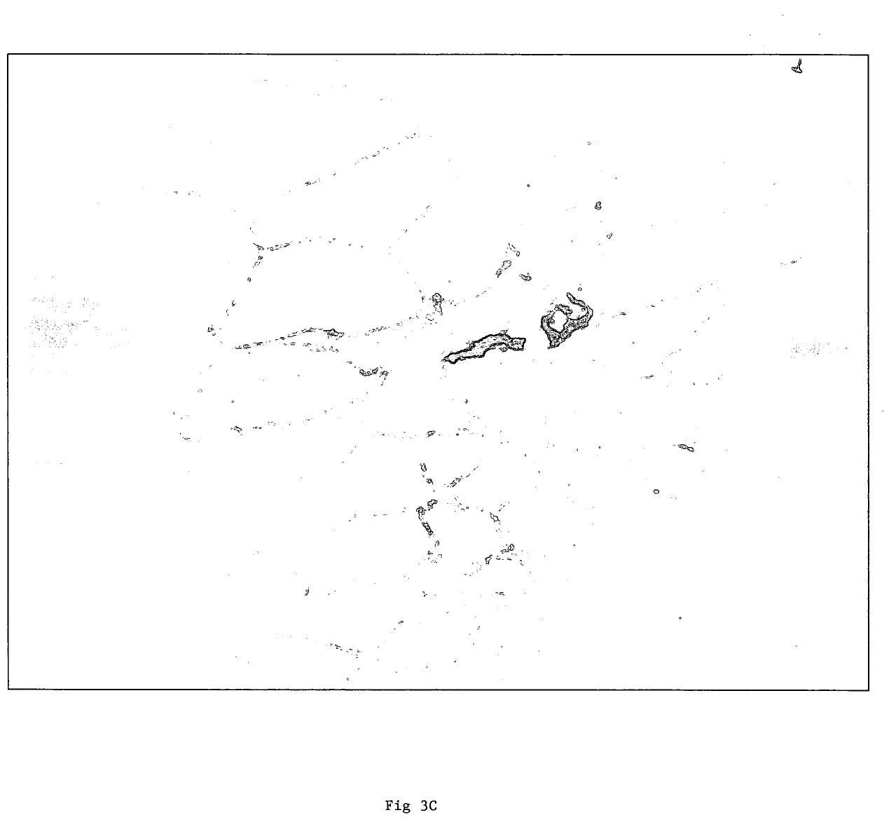

Immunocytochemistry results indicated that in Dp260, Tg+ mice, the Dp260 protein localized to the sarcolemma membrane. The DM mice had no dystrophin, and showed no localization (FIG. 3b). In eight-week-old DM/Tg+ mice, fluorescence intensity varied from cell to cell, as shown in FIG. 3a, but appeared more uniform and localized to cell membranes at sixteen weeks as shown in FIG. 3c.

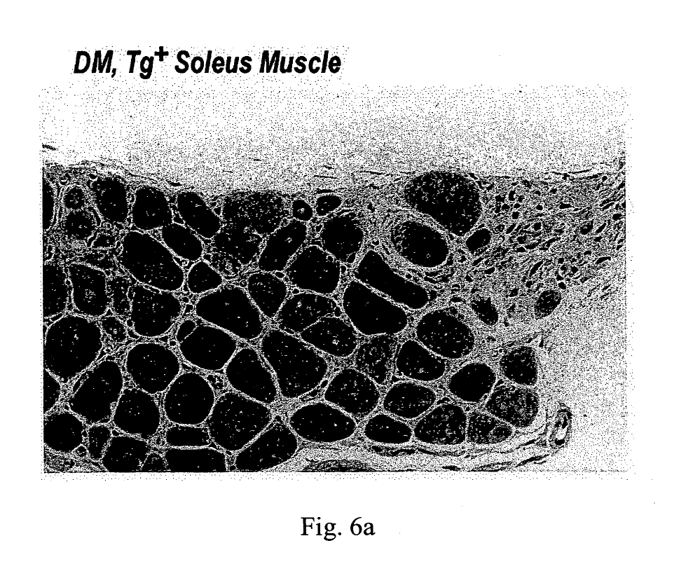

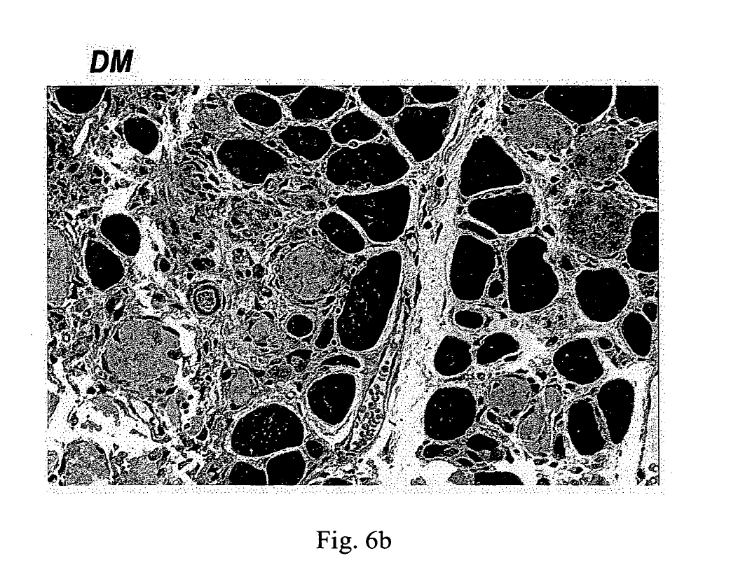

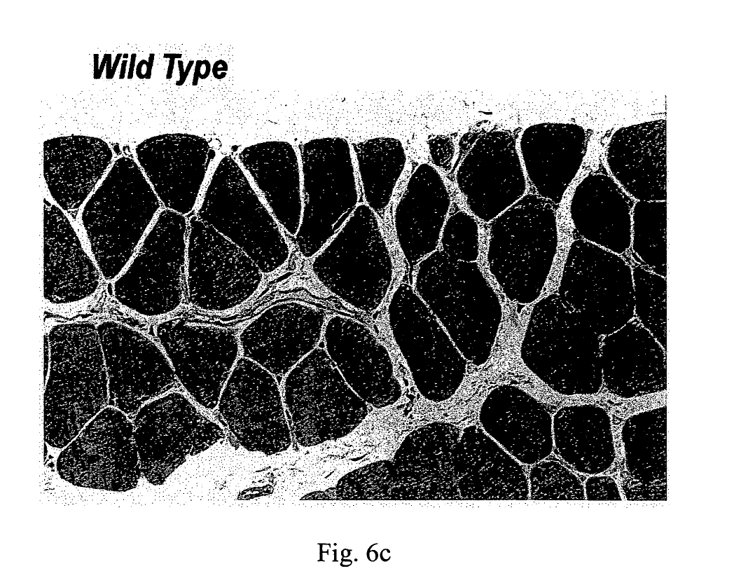

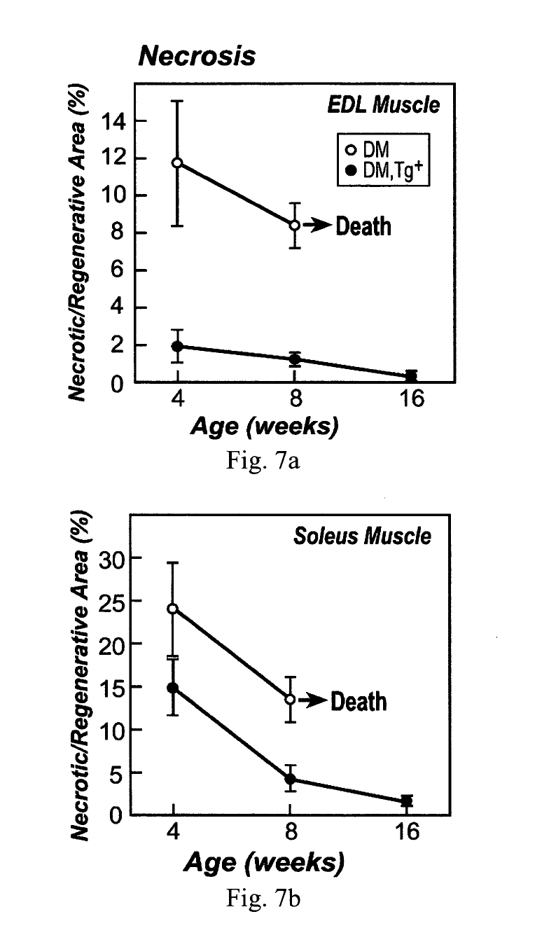

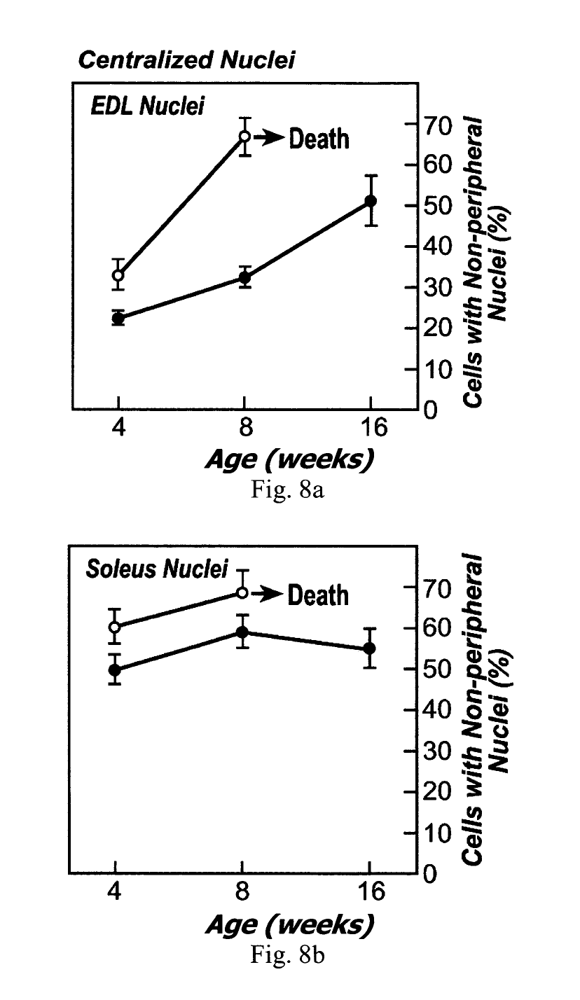

In histological analyses of muscles, the DM mice without the Dp260 transgene (FIG. 6b) showed extensive areas of muscle fiber degeneration, fibrosis, and infiltration by phagocytic cells, which indicates massive necrosis and inflammation of muscle tissue. This pathology is not completely eliminated by expression of the Dp260 transgene in DM/Tg+ mice, but the affected areas are much more focal and limited than those seen in DM mice. The appearance of the soleus muscles of the DM/Tg+ mouse was much closer to the morphology of the soleus of a wild-type age-matched control animal (FIG. 6c). Quantitative analysis shows that the percentage of necrotic areas for both types decreases with age, but by 16 weeks, DM/Tg+ mice have almost no necrosis in the EDL and soleus muscles, while DM mice have progressively more muscle necrosis until death. The percentage of muscle fibers with centrally located nuclei is a marker of chronic degeneration and regeneration in skeletal muscle. It increased with age in both DM and DM/Tg+ mice, but DM/Tg+ averages were significantly lower (p<0.05) than age-matched averages in both soleus and EDL muscles.

### EXAMPLE 5

### Magnetic Resonance Imaging (MRI) and Radiography

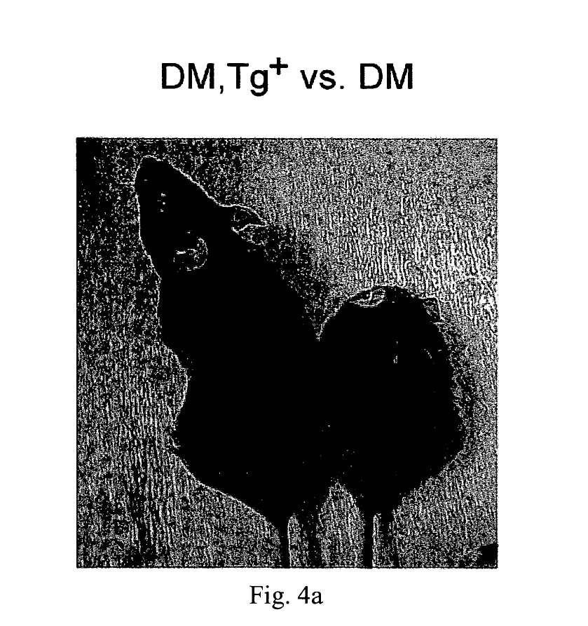

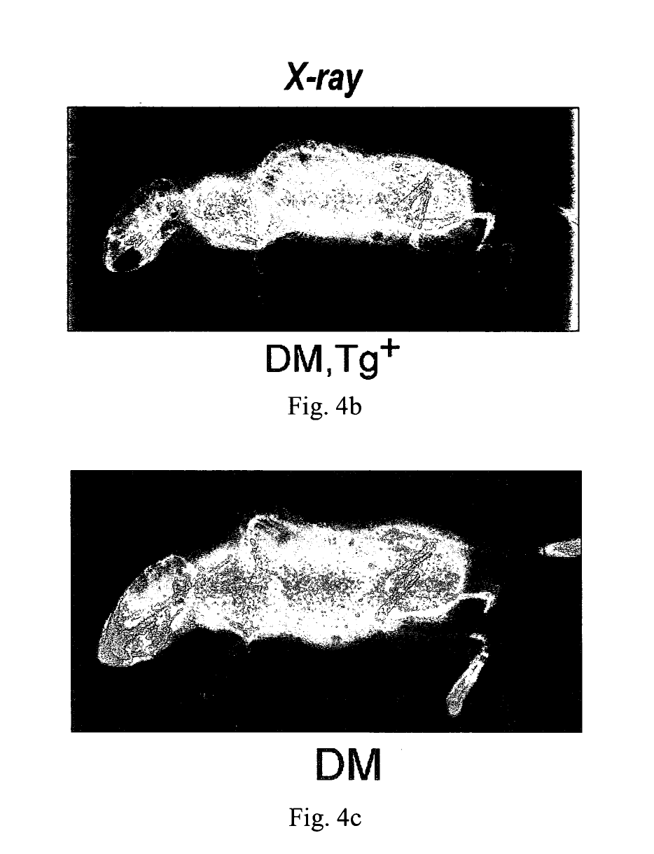

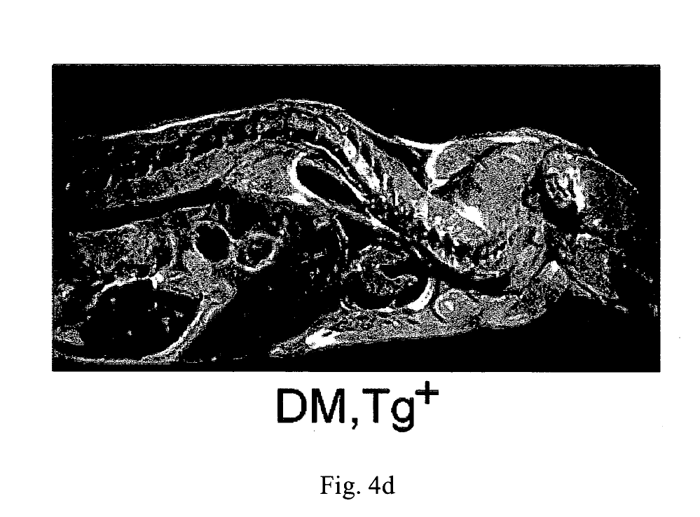

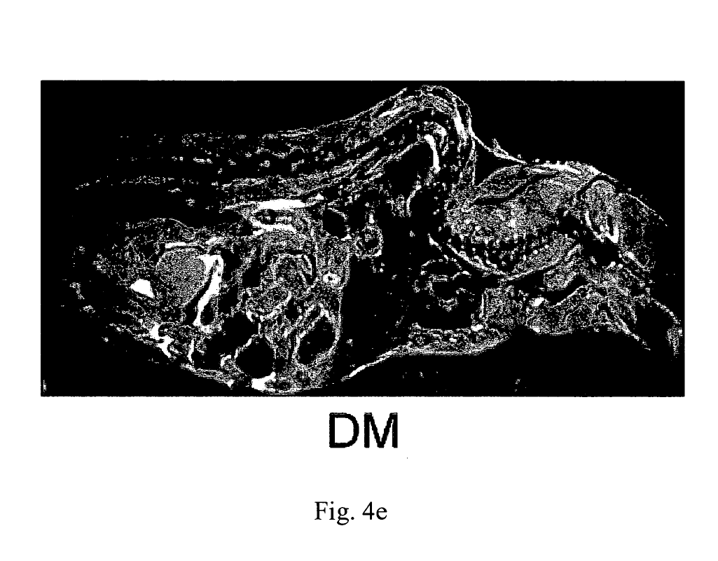

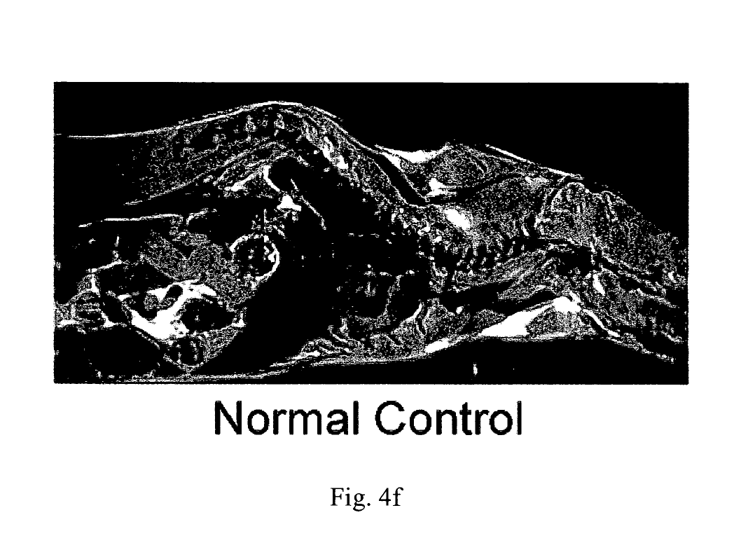

Sagittal MRIs were performed on DM, and DM/Tg+ mice on a horizontal bore 9.4 T Varian system using a mouse volume coil and a spin-echo imaging sequence with these parameters: TR/TE=2000/14 ms; Field of View=60×30 mm; image matrix=256×256 pixels; slice thickness=1 mm; and number of averages=2. MRI showed that severe disfigurement seen in DM mice (FIG. 4e) was not present in DM/Tg+ mice (FIG. 4d). DM mice also showed an apparent reduction in the thickness of both paravertebral muscle bundles and the myocardium as compared to wild type animals (FIG. 4f). These features in the DM/Tg+ animals were indistinguishable from wild type animals by MRI. The width of the heart muscle of DM/Tg+ mice seems to be thicker than that of the DM mice, and more comparable to that of the normal control mouse.

Kyphosis, the quadruped cognate of scoliosis seen in DMD, is characteristic of severely dystrophic DM mice. Radiographs, performed using standard methods, on 3 DM and 3 DM/Tg+ mice show the effect of human Dp260 expression on kyphosis in mice. The xray image shown in FIG. 4b shows the severely kyphotic spine of a DM mouse, the curvature of which measures 120° by goniometric analysis. In comparison, DM/Tg+ mice show spinal curvature of 56°, as seen in FIG. 4c, similar to that seen in normal mice.

### EXAMPLE 6

### Electromyography (EMG) Studies

Electromyographic responses to needle-electrode insertion were recorded in limb muscle from DM/Tg+ and DM mice using methods previously described by Carter et al. in Am. J. Phys. Med. Rehabil. 71:2-5 (1992) and Dumitru in Electrodiagnostic Medicine 2d Edition, 276-277. EMG studies were conducted in the tibialis anterior using a Neuromax EMG system (XL Tek, Ontario, Canada). Settings were standardized with a notch filter and adaptive filter both at 60 Hz, Low Frequency Filter at 30 Hz, High Frequency Filter at 10,000 Hz, gain at 200 mv/division, timebase at 10 ms/division, and negative trigger slope. The ground and reference electrodes were subcutaneously placed EEG subdermal recording needles (Nicolet 019-409700, Nicolet Biomedical, Madison, Wis.) that were monopolar needle electrodes with 0.25 mm² recording surfaces (TECA Corp., Ontario, Canada). All mice were anaesthetized with 0.6 mg/g weight of Avertin (tribromoethanol, Sigma). Weights were obtained at each EMG testing.

The presence of CRDs in EMG tests indicates muscle membrane instability and muscle pathology. To track CRDs, the muscle belly was divided into four equal quadrants and in four week intervals, recorded how many quadrants had CRDs, and how many CRDs (with insertional activity) there were in total. EMG activity was recorded in four directions, with needle advancements radiating outward from the center in approximately 0.5 mm increments. Four advancements were made in each quadrant, and the side of the animal studied was alternated for each 4 week interval to minimize trauma artifacts. The quadrants with CRDs were scored 0 to 4, and the CRD totals were scored 0 to 16.

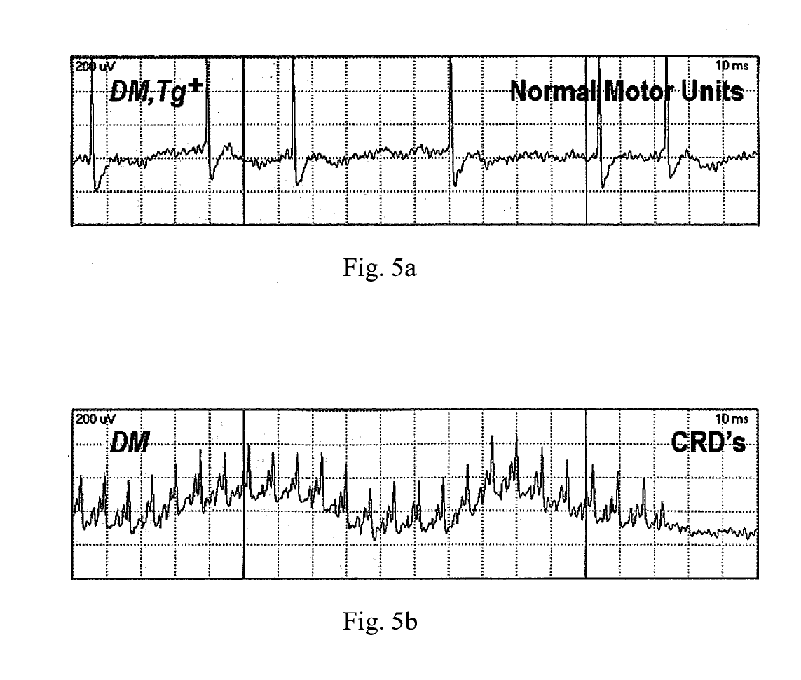

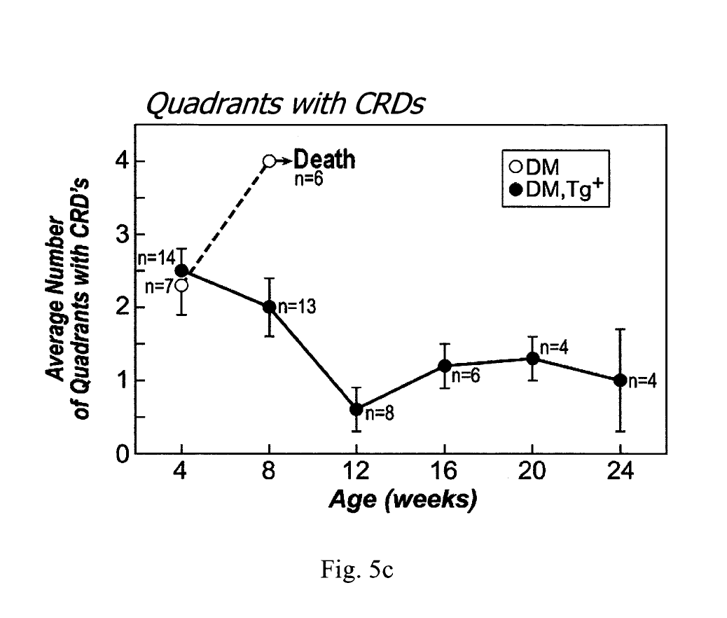

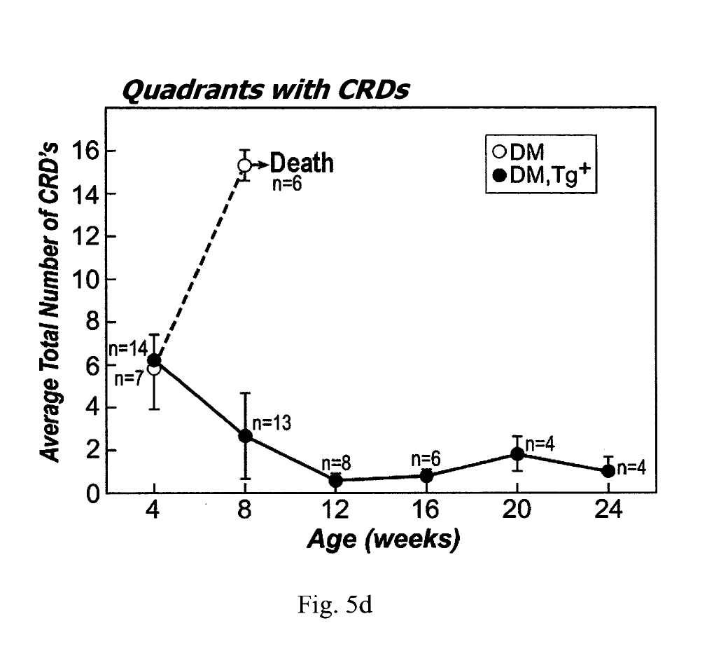

Electromyography directly assesses the muscle membrane stability and muscle pathology of DM and DM/Tg+ mice. Older DM/Tg+ mice show a normal EMG pattern with individual motor units firing (FIG. 5a). DM mice show a CRD pattern that typifies abnormalities in dystrophinopathies. CRDs are commonly seen in neuropathic conditions associated with muscle denervation and myopathic conditions. Electrophysiological responses were collected in the two mouse groups over time and were correlated with their clinical appearances. In four-week-old mice, there was no significant difference in the number of quadrants with CRDs or in the prevalence of total CRDs between the DM and DM/Tg+ groups of mice. (FIGS. 5c and 5d). As the mice aged, the DM mice had more quadrants with CRDs and higher total CRDs, while the DM/Tg+ mice had fewer of each. (FIGS. 5c and 5d). At eight weeks, the differences were significant, both for the number of quadrants with CRDs (p=0.002) (FIG. 5c), and for the total number of quadrants with CRDs (p<0.001) (FIG. 5c). CRDs were noted in all four quadrants of the DM mice, while CRDs were noted in fewer quadrants of the DM/Tg+ mice. The DM mice died between eight and twelve weeks, while the DM/Tg+ mice survived and were studied up to twenty-four weeks. Their EMG studies show that the number of quadrants with CRDs and the total number of CRDs decreased to a level typical of mild myopathy.

### EXAMPLE 7

### Mobility Studies

The locomotor activity data were recorded in a single force-plate actometer (obtained from Steve Fowler). The force plate actometer used a 12 cm by 12 cm sensing area. The spatial resolution was 1 mm and the temporal resolution was 0.02 s. The mice moved on an acrylic plastic surface roughened with fine sandpaper and the recording sessions lasted 15 minutes in a darkened, sound-attenuating room. Software written by Steve Fowler was used to log and analyze the data, which were analyzed by finding the average 95% confidence interval for the DM/Tg+ mice.

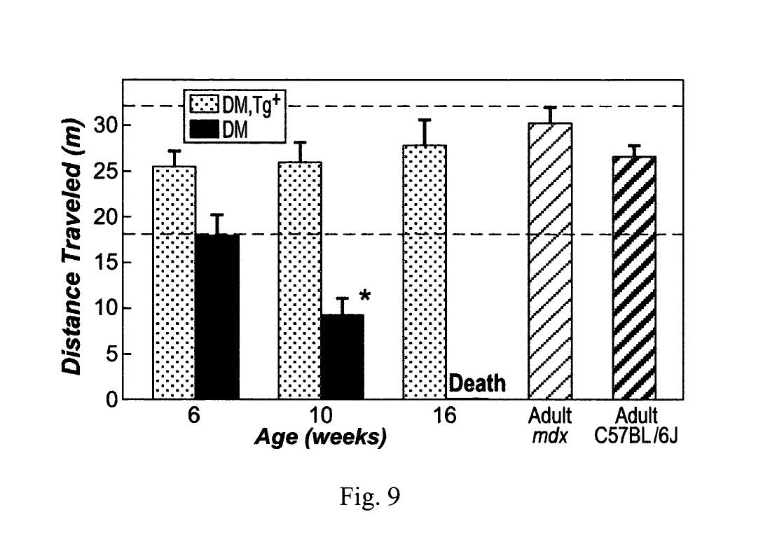

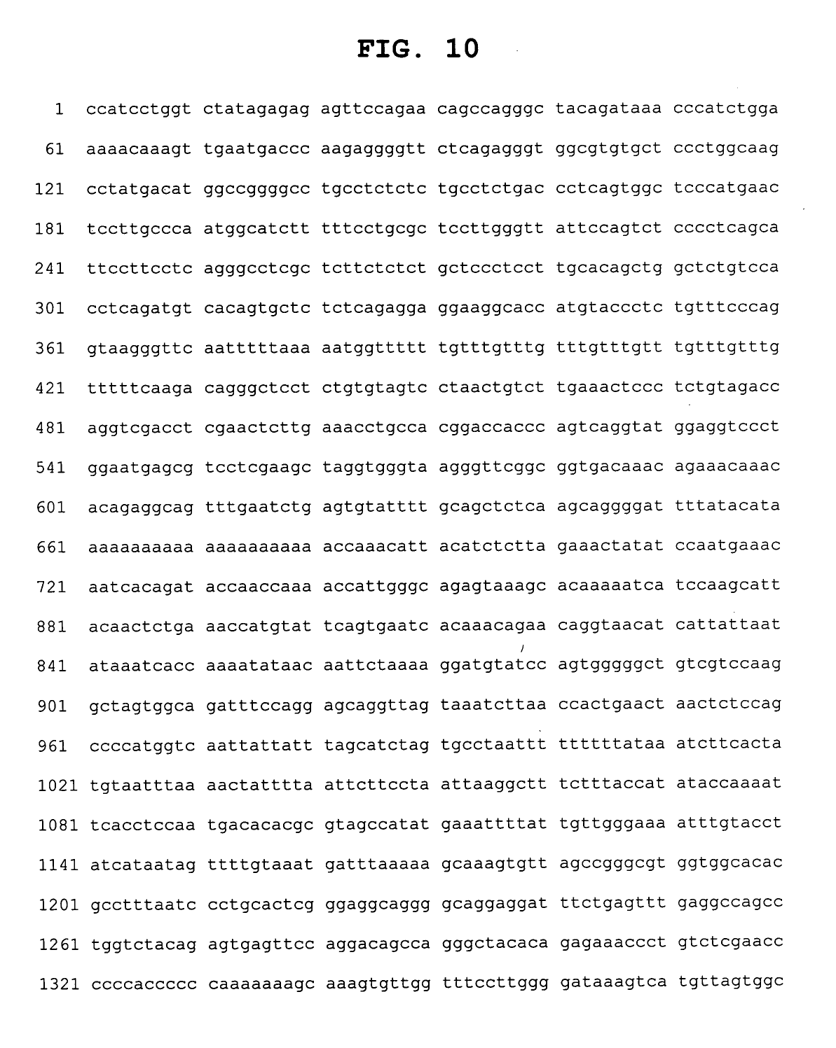

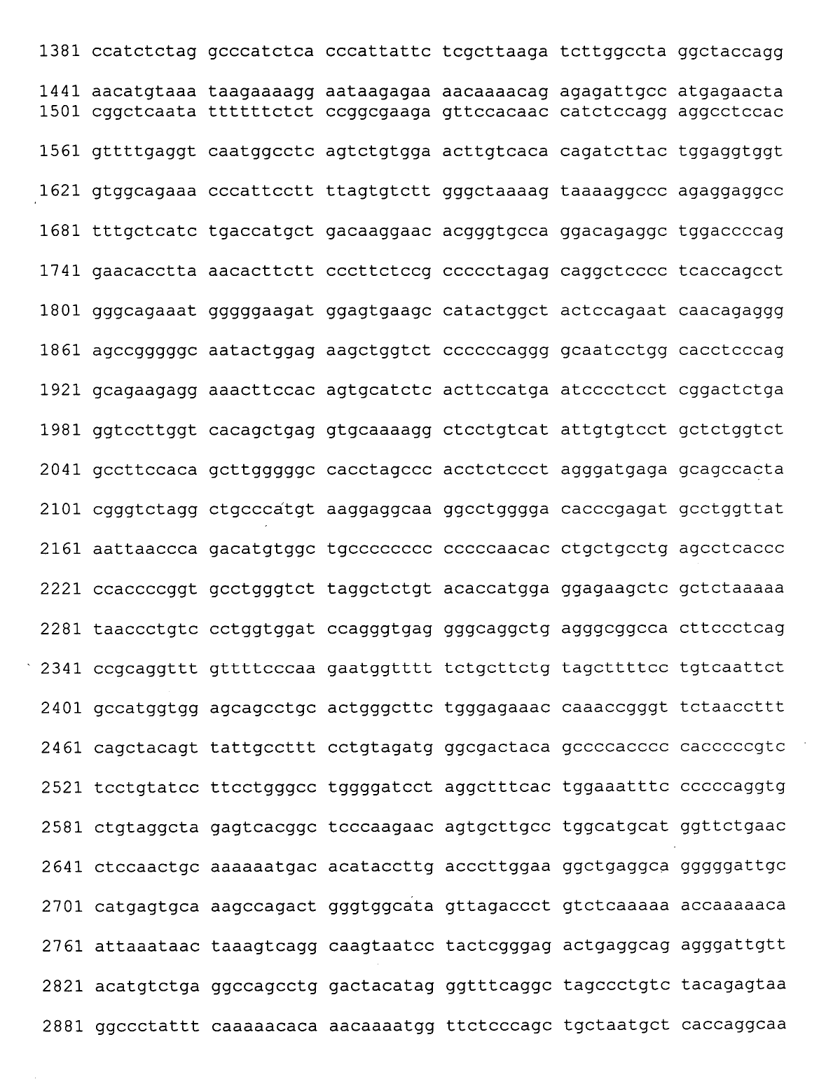

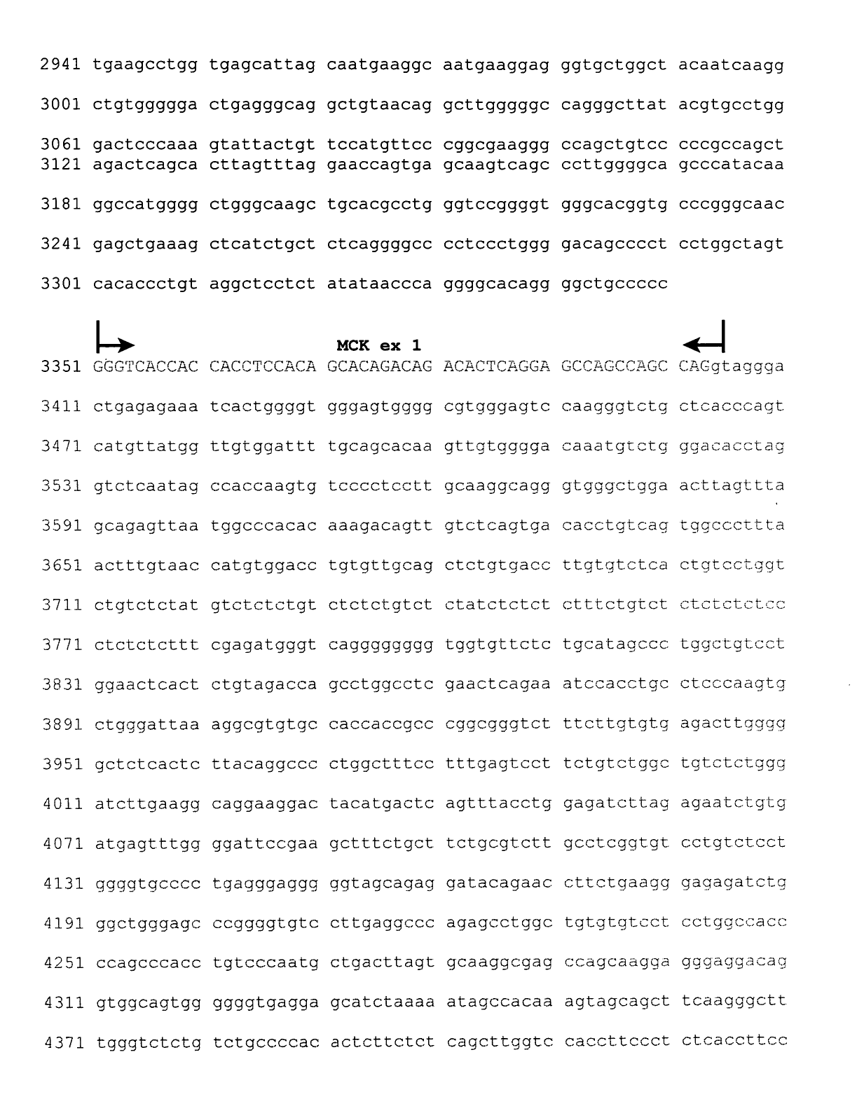

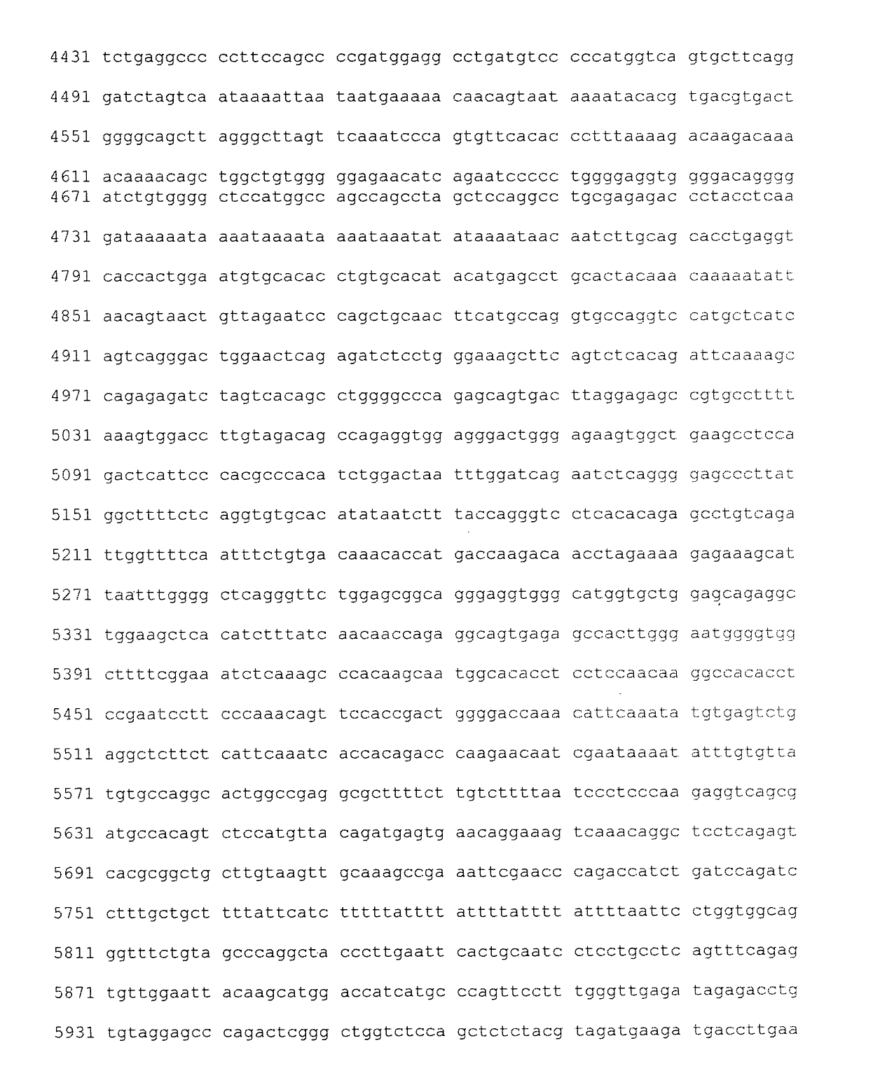

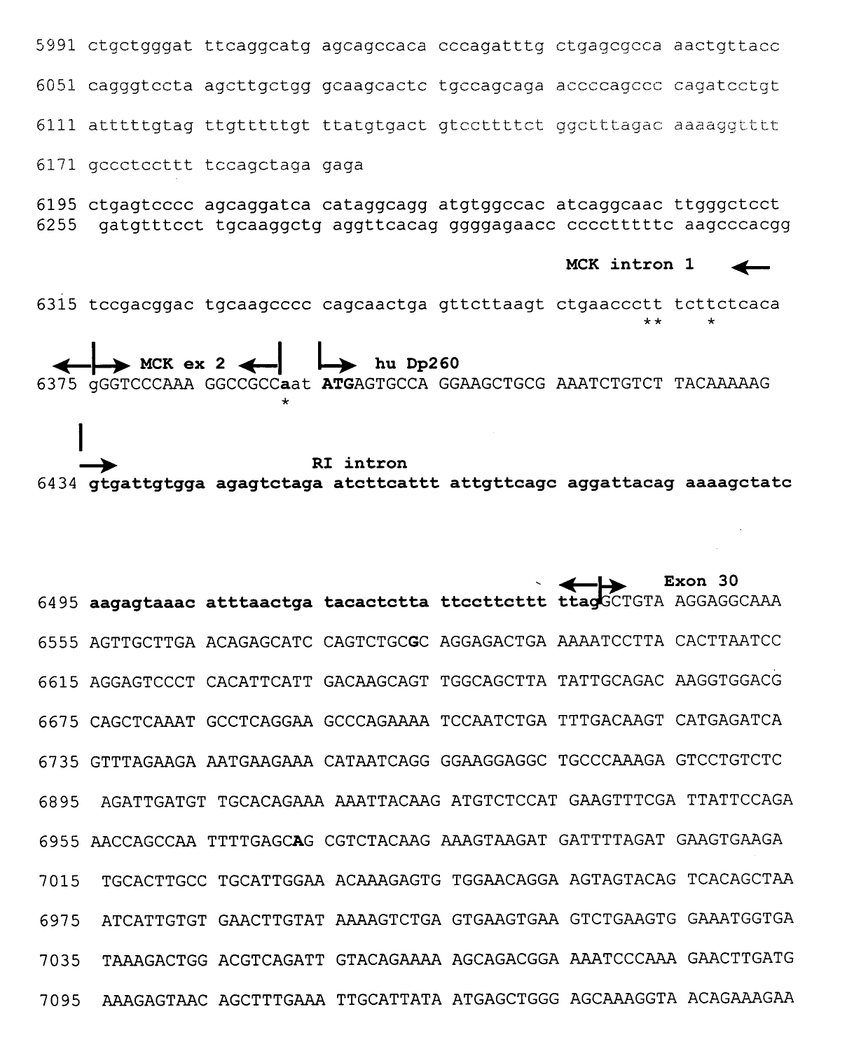

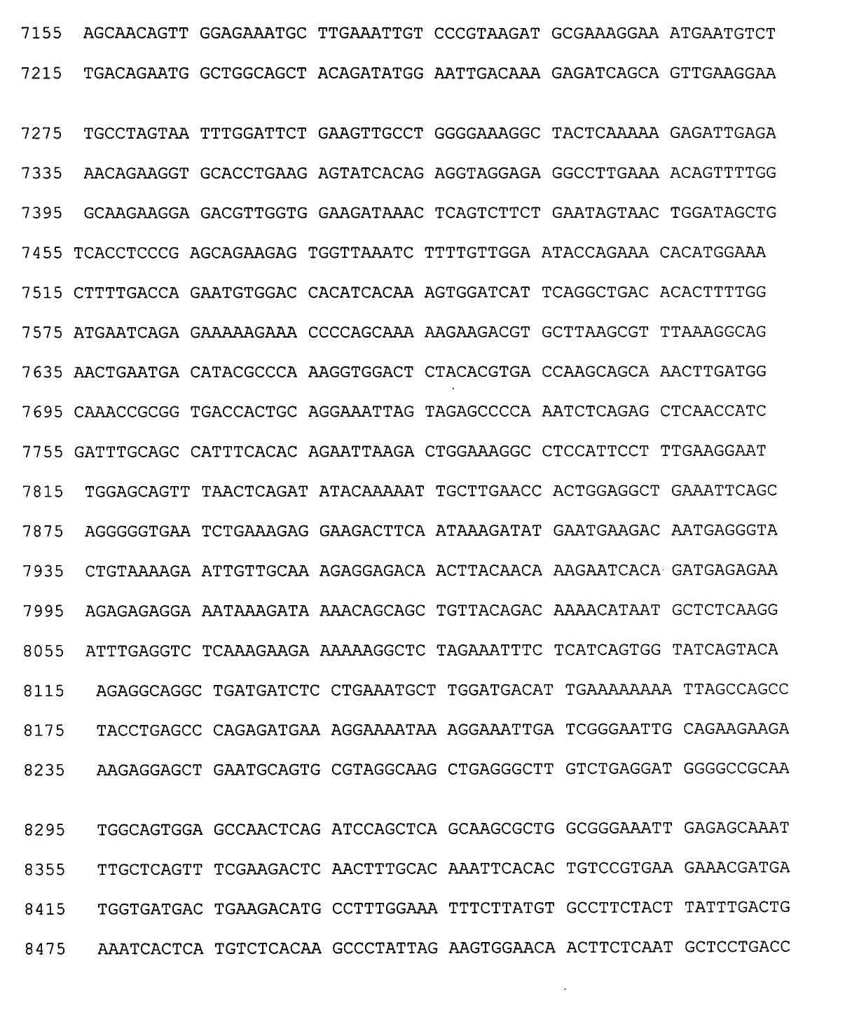

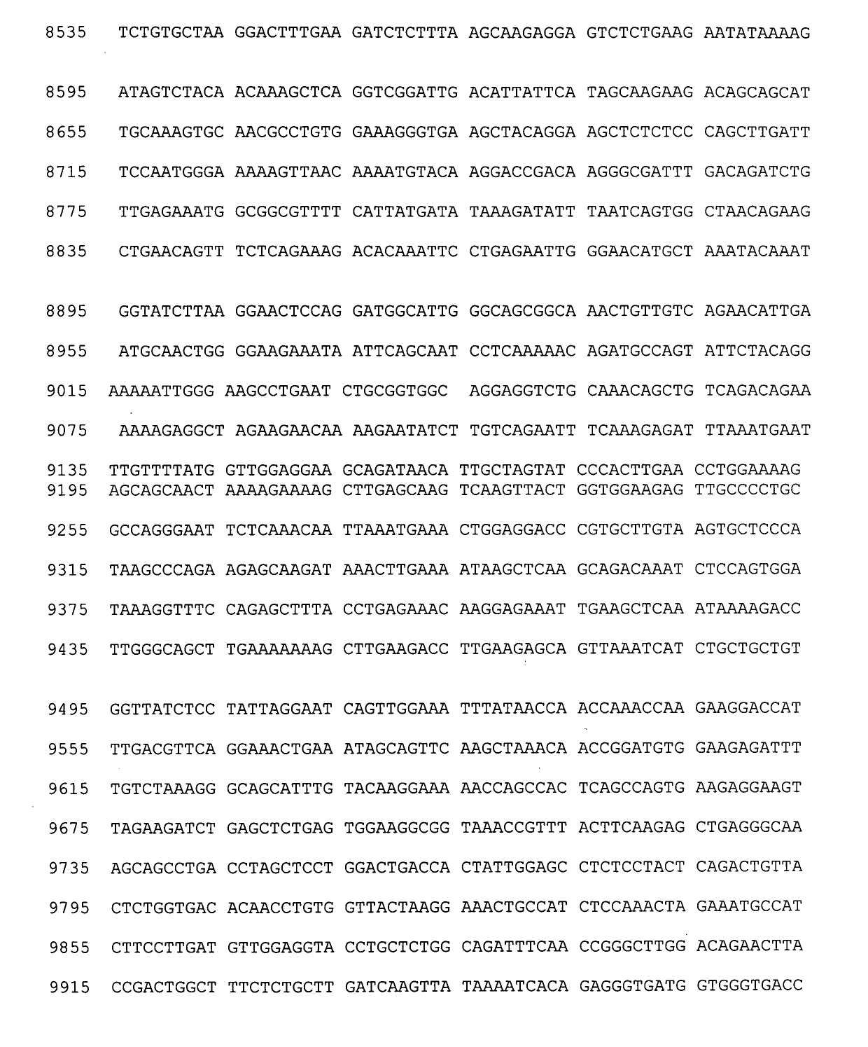

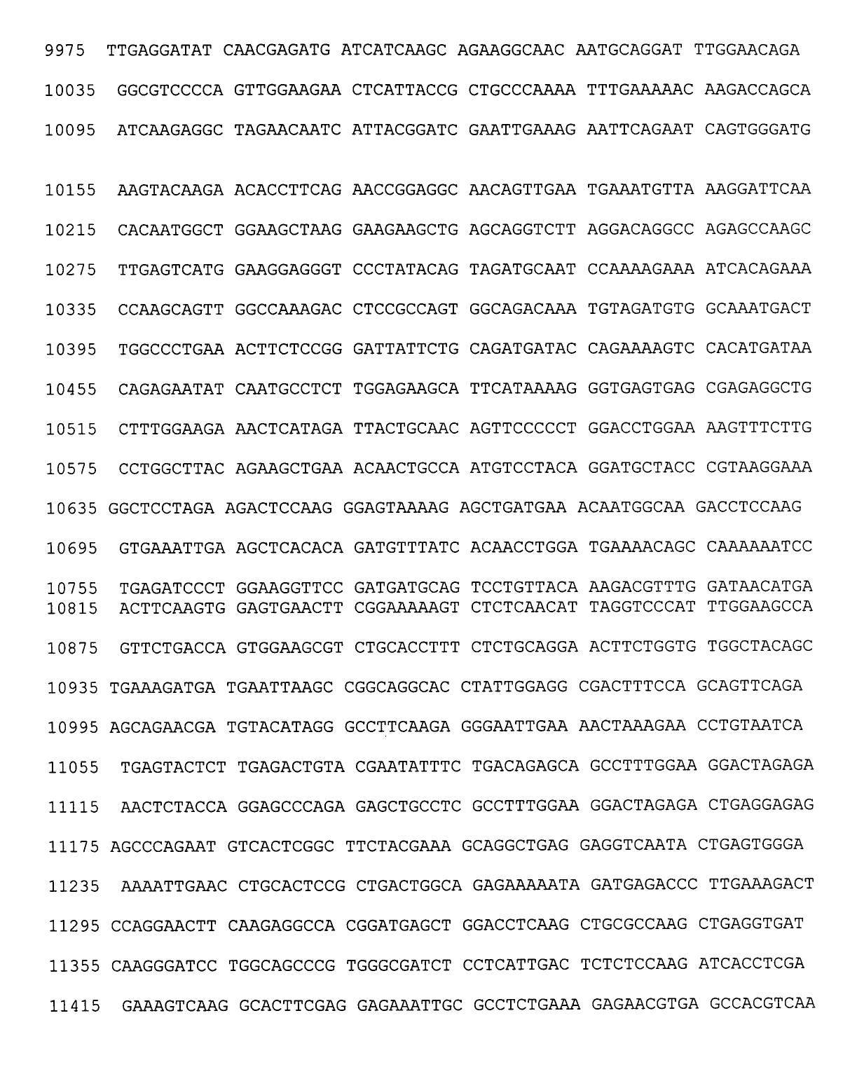

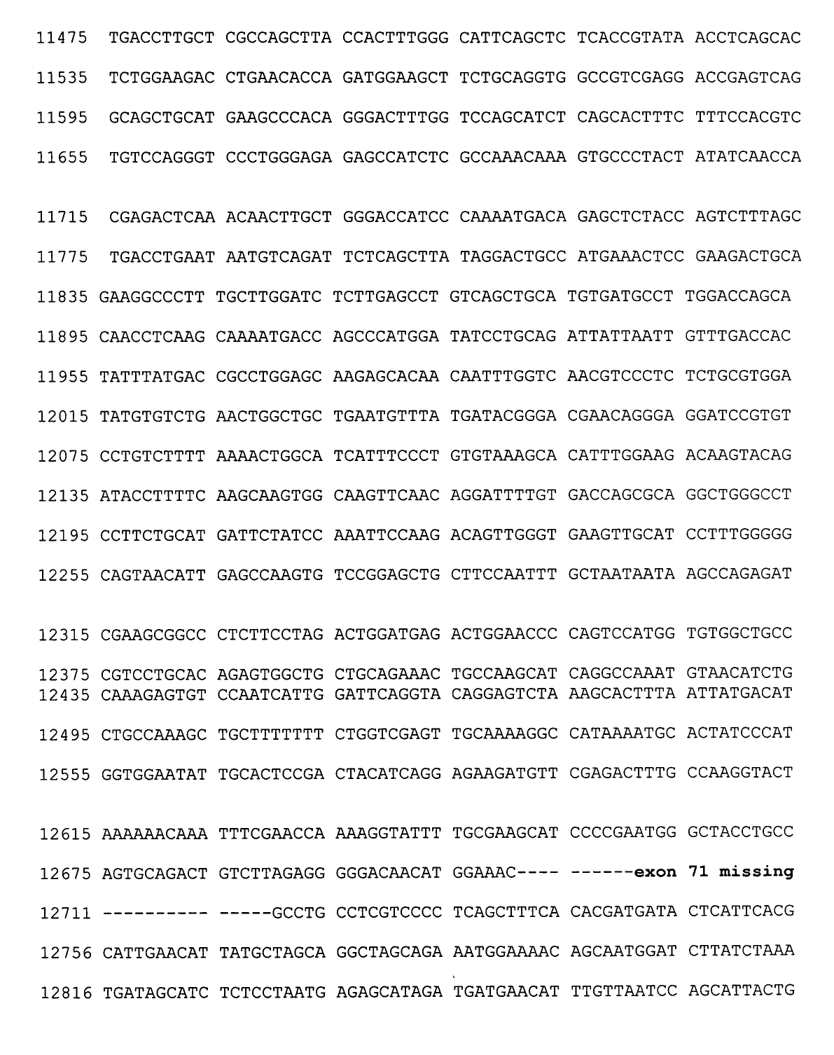

The force plate actometer measured the mobility of mice by their distance traveled. At six weeks, the DM mice moved less than the DM/Tg+ mice (FIG. 9), and the DM group data fell just outside the 95% confidence interval with the t-test showing marginal significance (p=0.07). At ten weeks, the DM mice were significantly impaired compared to the DM/Tg+ mice (p=0.002). The DM/Tg+ mice moved at levels comparable to untreated mdx mice and C57BL/6J control mice, which fall well within the 95% confidence interval for the DM/Tg+ mice. The DM/Tg+ mice also moved normally and appeared in generally good health, and did not show the decreased activity, abnormal, waddling gaits, and constracted, stiff limbs typical of DM mice.

### EXAMPLE 8

### Attentuation of Severe Muscular Dystrophy Phenotype in DM/Tg+ Mice

The severe muscular dystrophy phenotype seen in DM mice was improved in the DM/Tg+ mice, with the DM/Tg+ mice growing normally and living longer than the DM mice. The DM mice were undersized, where the DM/Tg+ mice grew normally (FIG. 4a). Clinical well being was measured by weight, because of its correlation with muscle mass and strength. At four weeks, the average body weight of DM/Tg+ mice [18.1±0.7 g (n=14)] was significantly larger (p=0.001) than the average body weight of DM mice [14.1±0.6 g (n=7)]. By eight weeks, the DM/Tg+ mice grew to 26.9±0.7 g (n=12) and by sixteen weeks, to 30.8±1.2 g (n=6). By eight weeks, the DM mice had grown only to 17.9±1.3 g (n=6) and died shortly thereafter.

In general, the DM/Tg+ mice increased their weights to normal levels correlated with age, while DM mice made minimal weight gains and died prematurely. All twenty-eight of the DM/Tg+ mice produced for a lifespan study have lived longer than the average age of death of the 30 DM mice (2.9±0.3 months). Twenty-three of the DM/Tg+ mice have lived beyond the age of six months, and only six of them have died. This 21% rate of attrition is normal in laboratory mice. Seven of the DM/Tg+ mice have reached the age of one year or older.

### EXAMPLE 9

### DMD Treatment by Cell Removal, Transfection, and Administration

Cells from mice, dogs, horses and humans are removed from the animal and stably transfected with a genetic insert coding for retinal dystrophin protein using conventional methods and as further described above. The stably transfected cells are then administered to an animal in order to reduce the severity of at least one clinical symptiom of DMD. Preferably, the cells are removed from and administered to the same individual animal as in an autologous transplant. Such a procedure is then repeated as necessary throughout the individual animal's lifetime. More specifically, bone marrow cells can be isolated and grown in culture under conditions that maintain stem cell plasticity. They are then transfected with lentivirus containing the Dp260 transgene with a selectable marker gene, i.e. neomycin resistance. Alternatively, electroporation can be used as a method for introduction of the transgene to bone marrow cells. This can be done with co-transfection with a selectable genetic marker: a second plasmid containing the neomycin resistance gene. After selecting cells in neomycin, they can be transplanted into a recipient.

I claim:

1. A transformed vector comprising:
a nucleic acid sequence coding for dystrophin protein, said nucleic acid sequence not being included in said vector prior to transformation.

2. The transformed vector of claim 1, said vector further including at least one regulatory element selected from the group consisting of promoters, enhancers, and poly A signal sites.

3. The transformed vector of claim 1, said nucleic acid sequence being a transgene capable of expressing said dystrophin protein.

4. The transformed vector of claim 1, said nucleic acid sequence having at least 80% sequence identity with SEQ ID No. 10.

5. The transformed vector of claim 1, said vector being selected from the group consisting of plasmids and viral vectors.

6. The transformed vector of claim 1, said dystrophin protein being retinal dystrophin protein.

7. A cell comprising:
a nucleic acid sequence inserted into the genome of cell and thereby transforming said cell, said nucleic acid sequence coding for dystrophin protein.

8. The cell of claim 7, said nucleic acid sequence having at least 80% sequence identity with SEQ ID No. 10.

9. The cell of claim 7, said nucleic acid sequence further including at least one regulatory element selected from the group consisting of promoters, enhancers, and poly A signal sites.

10. The cell of claim 7, said cell being selected from the group consisting of myoblasts bone marrow cells, and side population bone marrow cells.

11. The cell of claim 7, said dystrophin protein being retinal dystrophin protein.

12. A transgenic animal having an exogenous nucleic acid sequence stably integrated into its genome, said nucleic acid sequence coding for dystrophin.

13. The transgenic animal of claim 12, said animal being selected from the group consisting of mice, humans, dogs, and horses.

14. The transgenic animal of claim 12, said nucleic acid sequence having at least 80% sequence identity with SEQ ID No. 10.

15. The transgenic animal of claim 12, said nucleic acid sequence further comprising at least one regulatory element selected from the group consisting of promoters, enhancers, and poly A signal sites.

16. The transgenic animal of claim 12, said dystrophin being retinal dystrophin.

17. A transformed cell having therein the vector of claim 1.

18. A method of reducing the severity of at least one clinical symptom of Duchenne Muscular Dystrophy in an animal comprising the steps of:
introducing a genetic insert into the genome of said animal, said insert coding for dystrophin protein.

19. The method of claim 18, said insert being a nucleic acid having at least 80% sequence identity with SEQ ID No. 10.

20. The method of claim 18, said insert further including at least one regulatory element selected from the group consisting of promoters, enhancers, and poly A signal sites.

21. The method of claim 18, said dystrophin being retinal dystrophin.

22. The method of claim 18, said introducing step comprising the step of stably transfecting a vector into said genome, said vector including said genetic insert.

23. The method of claim 22, said vector being selected from the group consisting of plasmids and viral vectors.

24. The method of claim 18, said clinical symptom being selected from the group consisting of complex repetitive discharges, kyphosis, necrosis, slack posture, growth retardation, and severe muscle weakness.

25. The method of claim 18, said animal being selected from the group consisting of humans, mice, horses, and dogs.

26. A method of reducing the severity of at least one clinical symptom of Duchenne Muscular Dystrophy in an animal comprising the steps of:
administering cells to said animal, said cells being transfected with a genetic insert coding for dystrophin.

27. The method of claim 26, said method further comprising the steps of removing cells from said animal and transfecting said cells with said genetic insert prior to said administering step.

28. The method of claim 27, said transfecting step occurring through a vector or electroporation of naked DNA.

29. The method of claim 26, said dystrophin being retinal dystrophin.

30. The method of claim 26, said genetic insert having at least 80% sequence identity with SEQ ID NO. 10.

31. The method of claim 26, said cells being selected from the group consisting of myoblasts, bone marrow cells, and side population bone marrow cells.

32. A transgene comprising a nucleic acid sequence that expresses dystrophin protein.

33. The transgene of claim 32, said dystrophin being retinal dystrophin.

34. The transgene of claim 32, said nucleic acid sequence having at least 80% sequence identity with SEQ ID No. 10.

35. The transgene of claim 32, said nucleic acid sequence being derived from ATCC clones 57670, 57672, 57674, and 57676.

36. The transgene of claim 32, said nucleic acid sequence being derived from isoform resulting from alternative splicing of said dystrophin.

37. The transgene of claim 32, further comprising at least one regulatory element selected from the group consisting of promoters, enhancers, and poly A signal sites.
